(19) United States
(12) Patent Application Publication
(10) Pub. No.: US 2019/0383807 A1
(43) Pub. Date: Dec. 19, 2019

(54) METHODS AND SYSTEMS FOR CONCENTRATION OF SAMPLES FOR LATERAL FLOW ASSAYS

(71) Applicant: Tokitae LLC, Bellevue, WA (US)

(72) Inventors: John Thomas Connelly, Seattle, WA (US); Jeffrey L. Dantzler, Seattle, WA (US); Veronika A. Glukhova, Seattle, WA (US); Benjamin David Grant, Bellevue, WA (US); Stephen Paul Harston, Bothell, WA (US); Vedrana Ivezic, Seattle, WA (US); Bernhard Hans Weigl, Seattle, WA (US); Ozgur Emek Yildirim, Bellevue, WA (US)

(73) Assignee: Tokitae LLC

(21) Appl. No.: 16/006,984

(22) Filed: Jun. 13, 2018

(51) Int. Cl. G01N 33/543; G01N 1/40; B01L 3/00; G01N 33/569

(52) U.S. Cl. CPC G01N 33/54366; G01N 1/4077; B01L 3/5023; B01L 2400/0406; G01N 2001/4088; B01L 2300/0681; G01N 33/56933

(57) ABSTRACT

Methods and systems for capture concentration of analytes using lectins and other capture ligands are described. For example, stationary phase media functionalized with lectins are used for capture concentration and cleaning of TB lipoarabinomannan (TB LAM) prior to assay on a lateral flow assay (LFA) device, and filtration devices suitable for particulate or bulk capture media are described. Size-exclusion filtration is used to separate particles with captured analyte during washing and concentration steps. Captured analyte can be eluted from stationary phase media prior to application to a LFA or eluted directly onto a customized LFA device that includes a size-selective filter. In various aspects, a size-selective filter on a LFA is used to transfer particulate capture media on a LFA device.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### BACKGROUND

Lateral flow assay (LFA) devices are commonly used to perform assays to detect presence or absence, or in some cases quantity, of an analyte in a sample. For example, lateral flow assays are used to detect hormones indicative of pregnancy or ovulation, infectious disease vectors, and drugs of abuse and various other analytes, e.g., as discussed in U.S. Pat. No. 4,703,017 to Campbell et al.; M. Sajid, A.-N. Kawde, and M. Daud, (2015) “Designs, formats and applications of lateral flow assay: A literature review,” J. Saudi Chem. Soc., Vol. 19, pp. 689-705; and “Rapid Lateral Flow Test Strips: Considerations for Product Development,” Lit. No. TB500EN00EM Rev. C 12/13, © 2013, EMD Millipore Corporation, Billerica, Mass., each of which is incorporated herein by reference.

A sample fluid applied to a lateral flow assay device flows through the assay flow path in response to capillary forces, solubilizing and interacting with dried reagents as it moves though the assay flow path. The analyte of interest binds to a capture reagent immobilized in the assay flow path, resulting in a detectable signal indicating the presence or absence of the analyte. The signal may be visually detectable by a human to provide qualitative information regarding presence or absence of the analyte. Some lateral flow assays are sufficiently sensitive that it is possible to obtain quantitative information regarding the amount of analyte in the sample. In some cases, lateral flow assay devices are used in combination with hand-held or table-top readers. Lateral flow assays frequently are used to perform immunochromatographic tests such as sandwich assays or competitive binding assays, e.g. as discussed in U.S. Pat. No. 4,376,110 to David et al. and U.S. Pat. No. 4,855,240 to Rosenstein et al., both of which are incorporated herein by reference. Other types of assays, using different components for capturing and visualizing analytes of interest may be used instead, for example competitive binding assays, inhibition assays, or serum assays, e.g., as discussed in U.S. Pat. No. 4,703,017 to Campbell et al.; M. Sajid, A.-N. Kawde, and M. Daud, (2015) “Designs, formats and applications of lateral flow assay: A literature review,” J. Saudi Chem. Soc., Vol. 19, pp. 689-705, which is incorporated herein by reference.

The amount of sample that can be put on an LFA is often limited by the physical design of the LFA, rather than by the available sample volume. For example, the physical dimensions and absorptivity of the materials forming the LFA define the amount of fluid that can be handled by the LFA. Overall sensitivity of the LFA can be increased by concentrating samples to reduce the volume of fluid containing the analyte of interest. Another factor that limits the sensitivity of LFAs is interference from substances other than the target analyte that may be present in the sample. This interference can result in background signal from non-specific binding, reduction of detection signal to noise ratio, etc.

The capture, concentration, and washing approaches described herein make it possible to increase the sensitivity of various diagnostic assays, chiefly LFAs, by producing cleaner, more highly concentrated samples. Concentration of analytes (e.g. biomarkers) is achieved by preprocessing dilute samples to capture biomarkers from large-volume samples and performing filtration concentration to obtain a smaller volume sample suitable for application to a LFA. In an aspect, capture, concentration, and washing steps are performed separately from the assay, such that conditions for these steps can be optimized without being limited to conditions required for the assay itself. Originally collected samples (e.g. urine) containing analytes of interest may be highly variable in composition and may include additional components that produce background signal if applied directly to an assay. Washing and elution steps allow the analyte of interest to be delivered to the assay in a clean, concentrated sample from which undesired components have been removed, thus reducing background interference caused by undesired components.

In liquid phase capture methods, target antigens are usually first captured with nanoparticles conjugated (coated) with an antibody or other ligand in the liquid phase. Process parameters are optimized by adjustment of incubation time and/or agitation and mixing in order to overcome kinetic and mass transfer limitations and prevent clumping or aggregation. This is followed by suitable concentration and wash steps.

For example, “magnetic particle capture and elution” is a widely used platform. Magnetic beads functionalized with antibodies to an analyte of interest can be used to capture the analyte of interest, and magnetophoresis used to separate the magnetic beads and captured analyte from bulk liquid to concentrate the sample. However, because magnetic beads tend to clump they are not typically applied directly to a LFA. An elution step is often used to release the captured antigens from the magnetic beads, and the eluent is run on the LFA. However, using size exclusion filtration separation rather than magnetic separation results in fewer material and size requirements for capture beads. Magnetic beads are typically made of dense paramagnetic materials and need to be relatively large (˜<1 um) in order to exhibit sufficient magnetic dipole moment to be manipulated by external magnetic fields easily. In addition, the possibility to use smaller, less dense bead materials may reduce gravity settling issues during incubation and hence less need for mixing.

### SUMMARY

In an aspect, a sample filtration container includes, but is not limited to, a base defining a bottom of the sample filtration container; at least one side wall contiguous with the base, the at least one side wall enclosing an interior of the sample filtration container; an opening at a top of the sample filtration container, the opening adapted to receive a sample including a fluid component and a particulate material carried in the fluid component; a divider located within the interior of the sample filtration container and dividing the interior of the sample filtration container into an upper portion and a lower portion, the divider including a size exclusion filter, wherein the size exclusion filter has a first side communicating with the upper portion of the sample filtration container and a second side communicating with the lower portion of the sample filtration container, wherein the size exclusion filter has a pore size adapted to allow passage of the fluid component of the sample while blocking passage of the particulate material; and a capillary medium within the lower portion of the sample filtration container, the capillary medium adapted to draw the fluid component of the sample through the size exclusion filter from the upper portion to the lower portion of the sample filtration container. In addition to the foregoing, other aspects are described in the claims, drawings, and text forming a part of the disclosure set forth herein.

In an aspect, a lateral flow assay device includes, but is not limited to, a loading region adapted to receive a fluid containing a functionalized nanoparticle-captured analyte complex including one or more functionalized nanoparticle and an analyte of interest in a carrier fluid, the loading region including a sample pad; and a filter element overlying the sample pad, wherein the filter element includes pores small enough to block passage of the functionalized nanoparticle through the filter element but large enough to permit passage of the carrier fluid and unbound analyte of interest through the filter element to the sample pad; and a lateral flow membrane downstream of the sample pad and including one or more capture components adapted to capture the analyte of interest. In addition to the foregoing, other aspects are described in the claims, drawings, and text forming a part of the disclosure set forth herein.

In an aspect, a lateral flow assay device includes, but is not limited to, a support layer; an absorbent pad disposed on the support layer; a movable framework configured to fit closely and removably over the absorbent pad; a first filter element supported by the movable framework, the first filter element configured for fluid communication with the absorbent pad through one or more apertures in the movable framework, wherein the first filter element includes pores small enough to block passage of a functionalized nanoparticle-captured analyte complex through the first filter element but large enough to permit passage of a carrier fluid through the first filter element to the absorbent pad; a sample pad supported by the support layer, wherein the sample pad is configured so that the movable framework can be fit closely over the sample pad; and a lateral flow membrane downstream of the sample pad and including one or more capture components specific to the analyte of interest. In addition to the foregoing, other aspects are described in the claims, drawings, and text forming a part of the disclosure set forth herein.

In an aspect, a filtration-concentration device includes, but is not limited to, a filter membrane having a first side and a second side, the filter membrane having pores small enough to block passage of a functionalized nanoparticle-captured analyte complex from the first side to the second side but large enough to permit passage of fluid or unbound analyte from the first side to the second side; a housing configured to contain the filter membrane, the housing having an upstream chamber in fluid communication with the first side of the filter membrane and downstream chamber in fluid communication with the second side of the filter membrane; an inlet port in fluid communication with the upstream chamber, the inlet port adapted to receive a fluid sample containing a functionalized nanoparticle-captured analyte complex in a first volume of the fluid; a fluid outlet port in fluid communication with the downstream chamber, the fluid outlet port configured to permit fluid including a portion of the first volume of fluid to exit the filtration concentration device; and a retentate removal port in communication with the upstream chamber, the retentate removal port configured to allow removal of a retentate from the upstream chamber; wherein the filter membrane is chemically inert with respect to the functionalized nanoparticle-captured analyte complex and the fluid and exhibits little or no non-specific binding to materials in the fluid; wherein the upstream chamber has a volume sufficient to contain a second volume of fluid, wherein the second volume is less than the first volume. In addition to the foregoing, other aspects are described in the claims, drawings, and text forming a part of the disclosure set forth herein.

In an aspect, a capture concentration device includes, but is not limited to, a straight-walled container having an interior surface, a first end, a second end, and an opening at the first end, the straight-walled container adapted to receive a fluid sample including an analyte of interest and a fluid component; and a plunger including a sieve element configured to slidably engage with the interior surface of the straight-walled container and to support a stationary phase medium functionalized with at least one capture ligand adapted to bind an analyte of interest in the fluid sample, the sieve element having openings small enough to block passage of the stationary phase medium but large enough to permit passage of unbound analyte of interest and the fluid component; and a shaft attached to the sieve element and configured to transmit force to the sieve element to drive sliding movement of the sieve element within the straight-walled container. In addition to the foregoing, other aspects are described in the claims, drawings, and text forming a part of the disclosure set forth herein.

In an aspect, a TB LAM filtration device includes, but is not limited to, a stationary phase medium functionalized with at least one lectin adapted to bind a glycan of TB LAM to capture TB LAM from a fluid sample, the fluid sample including the TB LAM and a fluid component; and a sieve element having openings small enough to block passage of the stationary phase medium but large enough to permit passage of unbound TB LAM and the fluid component, wherein the sieve element is formed of a mildly hydrophilic, chemically inert material having minimal nonspecific binding to components of the fluid sample. In addition to the foregoing, other aspects are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1A, 1B, and 1C depict components used in analyte capture.

FIGS. 2A-2H depict a process for analyte capture and concentration with functionalized particles.

FIGS. 3A-3C illustrate a lateral flow assay device.

FIGS. 4A-4M depict a process for analyte capture and concentration with functionalized particles in a filter device.

FIGS. 5A-5C illustrate an alternative lateral flow assay device.

FIGS. 6A-6B depict use of a sample filtration container.

FIGS. 7A-7C depict alternative sample filtration containers.

FIG. 8 is a flow diagram of a method of using a sample filtration container.

FIG. 9 is a flow diagram of a method of detecting a biomarker of interest from fluid sample.

FIG. 10 is a flow diagram of a method of detecting a biomarker of interest from fluid sample.

FIG. 11 is a flow diagram of a method of detecting a biomarker of interest from fluid sample.

FIGS. 12A-12C illustrate a lateral flow assay device including a filter.

FIGS. 13A-13D illustrate a lateral flow assay device including a translatable component.

FIGS. 14A and 14B illustrate details of the lateral flow assay device of FIGS. 13A-13D.

FIG. 15A is a cross-sectional view of the structure of FIG. 14A taken at section line A-A.

FIG. 15B is a cross-sectional view of a structure lateral flow assay device.

FIG. 15C is a cross-sectional view of the structure of FIG. 15B taken at section line C-C.

FIGS. 16A-16D illustrate a lateral flow assay device including a hinge element.

FIGS. 17A and 17B illustrate details of the lateral flow assay device of FIGS. 16A-16D.

FIGS. 18A-18G depict a process for analyte capture and concentration with a functionalized membrane.

FIGS. 19A-19G depict a process for analyte capture and concentration with a functionalized stationary phase.

FIGS. 20A-20G depict a process for analyte capture and concentration with a capture concentration device.

FIG. 21 is flow diagram of a method of concentrating TB LAM.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

### Abbreviations and Definitions

As used herein, the abbreviations and terms listed below have the following meanings:

TB LAM—Mycoplasma lipoarabinomannan

LFA—lateral flow assay

Half strip LFA—an LFA strip that includes assay flow path (lateral flow membrane and capture chemistry) but does not include sample pad, conjugate pad, or functionalized particles.

Functionalized particles—nanoparticles or beads functionalized with capture antibodies, or some other ligand with affinity to target biomarkers or other analytes.

Methods and systems are described which relate to processing of a liquid sample containing a biomarker or other analytes to concentrate the sample prior to further processing, e.g. for detection of the biomarker on a lateral flow assay. For example, sample concentration can be used to improve the sensitivity and effectiveness of a diagnostic assay. In in aspect, methods and systems described herein are used to detect biomarkers (e.g. TB-LAM) that can be bound by lectins. In an aspect, lectins are used as capture ligands. In other aspects, any ligand having selective affinity to a target analyte can be used, including but not limited to antibodies.

In an aspect, concentration is achieved by capturing an analyte from a sample that includes a large volume of a fluid component, separating the captured analyte from the fluid component, and releasing the captured analyte by elution into a smaller volume of a secondary fluid component, which may be applied to a downstream assay. The captured analyte can be washed prior to elution to remove components of the sample that could interfere with detection or contribute background noise.

In various aspects, the analyte is an immunogenic antigen, and the downstream diagnostic assay is a lateral flow assay (LFA). However, methods and devices described herein can be used for pre-processing samples for non-LFA assays and with non-immunogenic biomarkers, provided they can be captured on some sort of suspended solid phase such as nanoparticles, and in some instances be released by use of suitable elution buffers. The foregoing invention addresses these problems by carrying out antigen conjugation to create functionalized particle-captured analyte complexes off-LFA in the bulk liquid phase, followed by filter concentration and wash steps. The output of this process is clean, highly concentrated functionalized particle-captured analyte complexes that can be put directly on a modified version of a half strip LFA. In cases where free biomarkers are needed, they can be eluted from the capture beads with a suitable release buffer, yielding purified and concentrated biomarkers which can then be put on a suitable assay such as an LFA.

Two broad categories of capture-release approaches can be used. In a first approach, a capture ligand is immobilized onto a freely dispersed carrier phase having a large total surface area, e.g. the carrier phase can include nano/micro beads or other particles. This approach enables easy dispersion of the capture phase into the entire volume of the sample, thus reducing mass transfer limitations. However, it requires additional steps to manipulate the freely dispersed phase, e.g. to separate and/or concentrate it. In a second approach, a capture ligand is immobilized in or on a stationary phase which resides in a fixed part of a device and the sample containing analyte is flowed through the stationary phase one or more times. Alternatively, in some aspects the sample containing analyte is incubated with the stationary phase in the absence of flow. In an aspect, the stationary phase includes an open pore capture bed, such as a sponge, and the capture ligand is immobilized in the 3-D bulk of the open pore capture bed. In another aspect, the stationary phase includes a thin membrane (e.g., a coarse filter) that permits flow through of the fluid component but not the carrier phase, and the capture ligand is immobilized on the 2-D surface of the thin membrane.

### Analyte Capture Using Particulate Capture Medium:

As noted above, in one approach, analyte of interest is captured using particles functionalized with capture ligands.

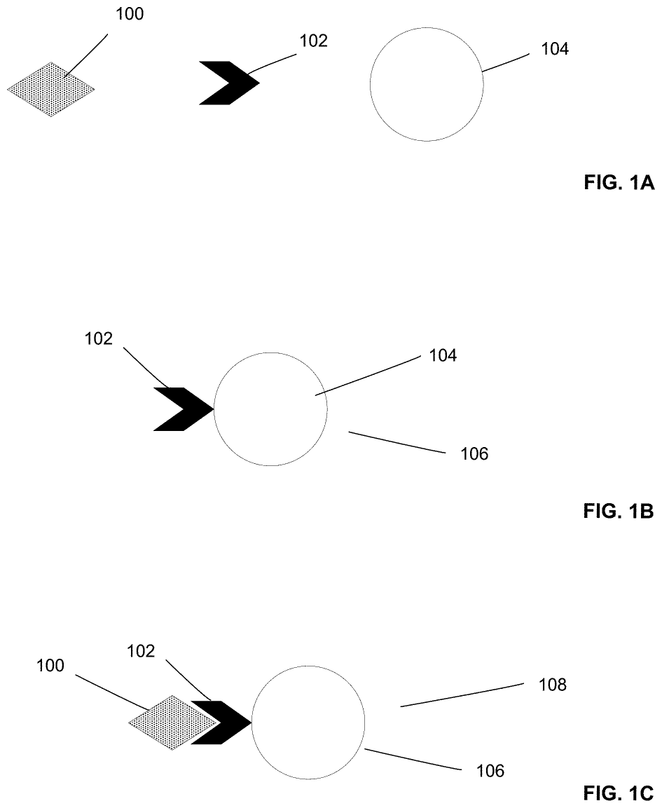

FIGS. 1A-1C illustrates in schematic form components used in capture of analytes. FIG. 1A depicts analyte 100, capture ligand 102, and particle 104. Analyte 100 is an analyte of interest that is to be isolated, detected and/or quantified from a fluid sample. In an aspect, analyte 100 is a biomarker. Capture ligand 102 is a ligand having specific binding affinity for analyte 100. Capture ligand 102 may be, for example, a lectin or an antibody. Particle 104 is a particle (e.g., a microbead, a microparticle, a nanoparticle, or a nanobead) that functions as a carrier phase. Particle 104 may be, for example, a latex, polystyrene, cellulose, e.g., NanoAct™ cellulose nanobeads (Asahi Kasei Corporation), glass, silica, gold, or gold-coated particle. In general, nanoparticles include particles having size typically specified in nanometers (indicating particle diameter) rather than molecular weight. For example, in various aspects, nanoparticles have diameters of between about 10 nm and 800 nm, between about 20 nm and 400 nm, between about 20 nm and about 100 nm, between about 100 nm and about 400 nm, between about 100 nm and about 200 nm, or between about 200 nm and about 400 nm. In various aspects, particles are suitable for use in lateral flow assay or other bead-based assays. As shown in FIG. 1B, in an aspect, particle 104 is functionalized with capture ligand 102 to form functionalized particle 106. As shown in FIG. 1C, analyte 100 can bind to functionalized particle 106 to form functionalized particle-captured analyte complex 108.

### General Method for Analyte Capture Using Particulate Capture Medium

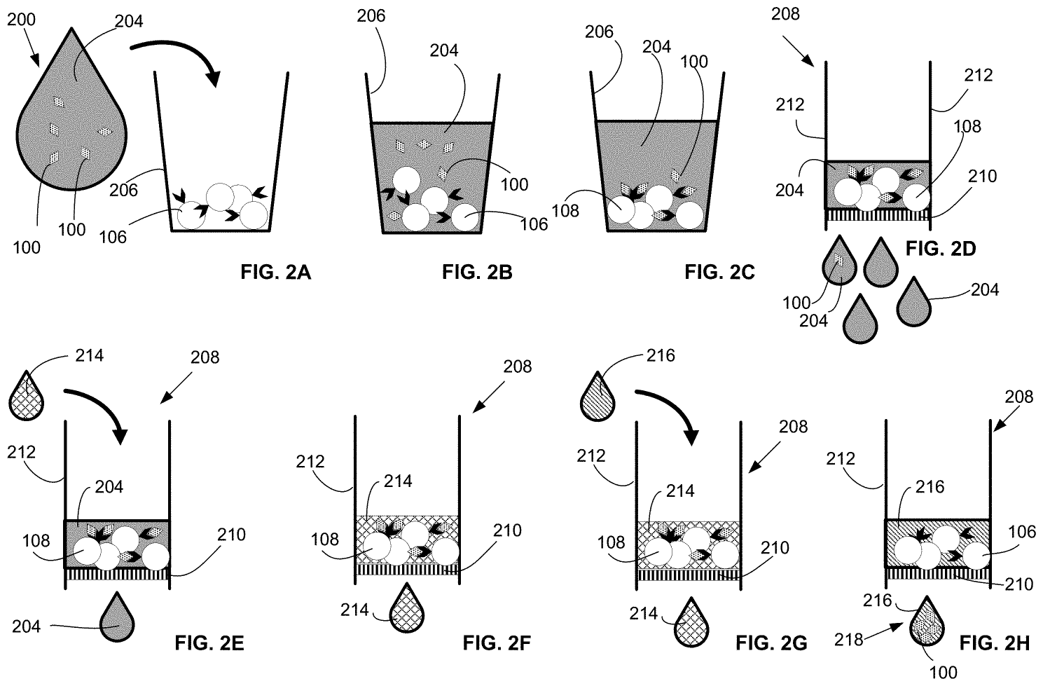

FIGS. 2A-2H illustrate a generalized method for capture and concentration of an analyte. In FIG. 2A, a volume of a sample 200 containing an analyte 100 is collected. In an aspect, the sample is large (e.g. 20 ml) relative to the volume of fluid that can be readily analyzed on a lateral flow assay (e.g. 100 μl). Sample 200 includes analyte 100 in fluid component 204. Sample 200 is collected in sample container 206 (e.g., a sample collection cup) and mixed with functionalized particles 106, as shown in FIG. 2B. Functionalized particles 106 are as described in connection with FIGS. 1A-1C. In an aspect, functionalized particles 106 are present in sample container 206 prior to collection of sample 200. In other aspects, functionalized particles 106 are added to sample container 206 at the same time as sample 200, or subsequent to addition of sample 200. Sample 200 is incubated with functionalized particles 106 for an incubation period and under an incubation condition sufficient to produce binding of analyte 100 to the functionalized particles to produce functionalized particle-captured analyte complex 108, as depicted in FIG. 2C. By performing the capture steps in FIGS. 2B-2C prior to applying the sample to an LFA, the volume of sample that can be exposed to the functionalized particles is much greater than the volume that could be put on an ordinary LFA. Accordingly, as much of the dilute analyte as possible is captured on the functionalized particles to form functionalized particle-captured analyte complexes, and the volume of sample that can be processed is not constrained by the specific design of the LFA. Various incubation parameters can be selected to optimize complex formation (e.g., incubation time, presence of agitation/mixing, buffer chemistry, etc.). It will be appreciated that by performing the incubation prior to applying the sample to the LFA, there are fewer constraints on how the incubation is performed.

Once the functionalized particle-captured analyte complexes are formed within the fluid volume including the original sample combined with the functionalized particles, they are concentrated down to a much smaller volume that is compatible with the LFA. This is accomplished by size exclusion filtration that retains the functionalized particle-captured analyte complexes but allows passage of the remaining fluid component. A concentrated dispersion of functionalized particle-captured analyte complexes suspended in a small amount of residual fluid component is obtained. As shown in FIG. 2D, fluid component 204, containing functionalized particle-captured analyte complex 108, as well as any unbound analyte 100 and/or unbound functionalized particles 106 is transferred to separation structure 208. Separation structure 208 includes filter 210 which has a pore size large enough to permit passage of fluid component 204 and unbound analyte 100 but not functionalized particle-captured analyte complex 108. Accordingly, separation structure 208 is used to separate functionalized particle-captured analyte complex 108, as well as any unbound functionalized particles 106 (not shown in FIG. 2D) from fluid component 204, and unbound analyte 100 carried in fluid component 204. In an aspect separation structure 208 also includes walls 212 which help to contain fluid component 204. As shown in FIG. 2D, a small amount of fluid component 204 may remain with functionalized particle-captured analyte complex 108 in separation structure 208 after separation, but the amount of analyte (bound in functionalized particle-captured analyte complex 108) within separation structure 208 is significantly concentrated relative to in the original sample 200. The fluid component in which the functionalized particle-captured analyte complexes are suspended may contain background producing substances that cause interference and negatively affect the assay sensitivity. Accordingly, in some aspects it is desirable to perform a wash step. This may be done by adding a volume of a compatible buffer to the functionalized particle-captured analyte complexes and again perform size exclusion filtration to retain the functionalized particle-captured analyte complexes on the filter while wash buffer along with any background producing substances passes through the filter and can be discarded. FIG. 2E depicts a wash step in which wash buffer 214 is added to separation structure 208, to wash away remaining fluid component 204 from functionalized particle-captured analyte complex 108. One or more wash steps can be carried out, with excess wash buffer 214 passing through filter 210, leaving functionalized particle-captured analyte complex 108 surround by a small amount of residual wash buffer 214, as depicted in FIG. 2F. In FIG. 2G, a small amount of elution buffer 216 is added to separation structure 208, to replace wash buffer 214. Elution buffer 216 is incubated with functionalized particle-captured analyte complex 108 for an elution period and under an elution condition sufficient to elute analyte 100 from functionalized particle 106, such that, as shown in FIG. 2H, elution buffer 216 containing analyte 100 can pass through filter 210 and be separated from functionalized particle 106, which remains in separation structure 208.

Concentrated sample 218, made up of analyte 100 in elution buffer 216, is significantly concentrated relative to the originally collected sample 200. Concentrated sample 218 is clean and in a small volume suitable for application to a lateral flow assay or other analysis device.

It will be appreciated the relative sizes of the first volume of sample 200 and the second volume of concentrated sample 218 determine how much the sample is concentrated. For example, if the first volume of sample fluid is twice the second volume of elution buffer, the concentration of TB LAM in the elution buffer will be twice the concentration of TB LAM in the sample fluid.

The total number of molecules=V₁C₁=V₂C₂

Where V₁=volume of the body fluid sample containing TB LAM, C₁ is the concentration of TB LAM in the body fluid sample, V₂ is the volume of elution buffer containing eluted TB LAM, and C₂ is the concentration of TB LAM in elution buffer.

The following equation can be used to describe the concentration obtained:

$${C}_{2} = {C}_{1} \frac{{V}_{1}}{{V}_{2}}$$

As an example, if the body fluid sample is urine, a typical sample size is roughly 10 ml to roughly 50 ml. It may be possible to load up to 5 ml of fluid onto a lateral flow assay, but it is more typical that lateral flow assays can accommodate smaller samples, e.g. between about 50 μl and about 400 μl, or between about 100 μl and about 200 μl. Thus, it is desirable to concentrate an analyte from a urine sample anywhere from about 50-fold to about 500-fold.

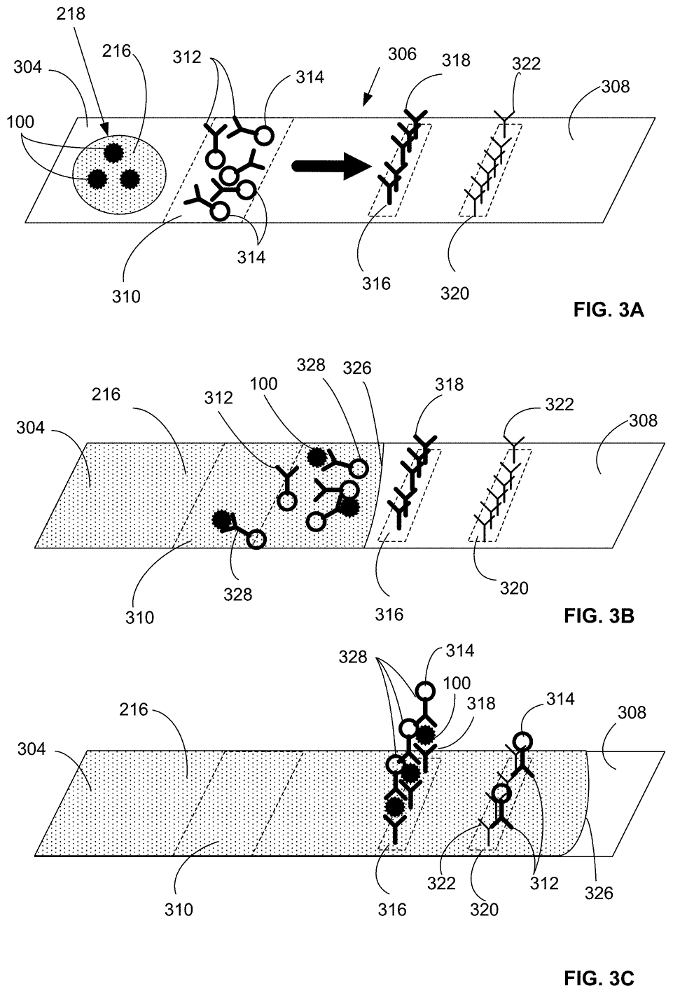

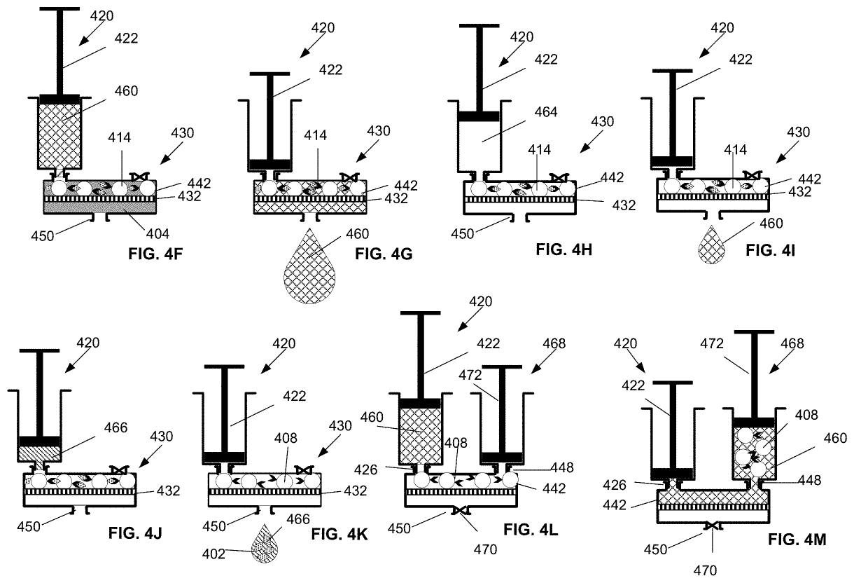

In some aspects, analytes are eluted into an elution buffer, as shown in FIGS. 2A-2H, to produce a concentrated sample 218 that can be applied to an assay as described in connection with FIGS. 3A-3C. Alternatively, as will be described in greater detail in connections with e.g. FIGS. 5A-5C, functionalized particle-captured analyte complex 108 can be removed from separation structure 208 and applied directly to a downstream assay. Such an approach is dependent on the use of particles suitable for application to a LFA or other bead-based assay.

FIGS. 3A, 3B, and 3C illustrate an example of processing of concentrated sample 218 (obtained through the approach illustrated in FIGS. 2A-2H) in a sandwich assay performed in a lateral flow format. In FIG. 3A, a concentrated sample 218 containing analyte of interest 100 in elution buffer 216 is applied at first end 304 of lateral flow assay device 306, and moves via capillary forces to second end 308, in the direction indicated by the heavy black arrow. Lateral flow assay device 306 includes a conjugate region 310 containing conjugate antibodies 312 specific to analyte 100. Conjugate antibodies 312 are conjugated to one or more detectable component 314, which may be, for example, latex beads, colloidal gold particles, other colloidal metals, colloidal carbon, fluorescent or luminescent labels, quantum dots, upconverting phosphores, bioluminescent markers, enzymes, magnetic or paramagnetic particles, dyes, electroactive compounds, or other suitable labels or markers. (Peter Chun, “Chapter 5. Colloidal Gold and Other Labels for Lateral Flow Immunoassays”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500ENOOEM Rev. C 12/13, © 2013, EMD Millipore Corporation, Billerica, Mass., both of which are incorporated herein by reference). In an aspect, detectable components are contained within and subsequently released from liposomes. Lateral flow assay device 306 also includes a test line 316, containing antibodies 318 immobilized on the material forming the assay flow path, and a control line 320 containing antibodies 322, which are specific to conjugate antibodies 312.

FIG. 3B depicts lateral flow assay device 306 after sufficient time has passed for elution buffer 216 to spread to fill first end 304 of lateral flow assay device and travel downstream through lateral flow assay device 306, to the location of flow front 326. As elution buffer 216 passes conjugate region 310, it solubilizes conjugate antibodies 312 and interacts with them to form bound conjugate antibodies 328, which are bound to analyte 100. Some of conjugate antibodies 312 remain unbound.

FIG. 3C depicts lateral flow assay device 306 after flow front 326 of elution buffer 216 has travelled to second end 308 of lateral flow assay device 306. As elution buffer 216 travels past test line 316, analyte 100 with bound conjugate antibodies 328 is bound by antibodies 318. Unbound conjugate antibodies 312 bind to antibodies 322 at control line 320. Detectable component 314 conjugated to bound conjugate antibodies 328 at test line 316, and unbound conjugate antibodies 312 at control line 320, produces a detectable signal that indicates presence of analyte 100 (at test line 316) and presence of properly functioning assay components (at control line 320), respectively.

It will be appreciated that for operation of the assay as depicted in FIGS. 3A-3C, elution buffer 216 is preferably suitable for eluting analyte 100 from functionalized particle 106 (in FIG. 2H) and also compatible with the detection chemistry on the lateral flow assay device 306. As an alternative, if the elution buffer is not optimized for the assay chemistry, an additional running buffer having properties better suited to the assay chemistry may be applied to the lateral flow assay device for moving analyte 100 through the lateral flow assay device to test line 316.

All diagnostic assays have constraints relating to the nature and the volume of the sample that is introduced to them, as well as the manner in which they are introduced. For example, in a typical lateral flow assay, a small amount of liquid sample (concentrated sample 218 in FIGS. 3A-3C), having a volume of between about 50 μl and about 100 μl, is applied to a sample pad at first end 304, sometimes followed by a running buffer. Sample then wicks along the device, encountering different regions along the way. A typical LFA contains nanoparticles (detectable component 314) functionalized with conjugate antibodies 312 specific to the target analyte being tested. If the target analyte is present in the sample, it is captured onto the functionalized nanoparticles to form functionalized particle-captured analyte complexes. Nanoparticles carrying the target antigens then flow downstream and are captured and concentrated at test line 316. A suitable signal (visual contrast, fluorescence, thermal response etc.) that is generated in the presence of functionalized particle-captured analyte complexes at the test line is measured/observed to establish the presence of the analyte in the original sample.

Syringe Filter Concentration Device for Use with Particulate Capture Medium

As noted herein above, one approach to obtaining concentrated, purified analyte, as depicted generally in FIGS. 2A-2H, is to capture it with functionalized beads, wash the functionalized particle-captured analyte complexes to remove extraneous materials while bound to the beads, and elute the analyte from the beads so that washed, concentrated, unbound analyte can be applied to an LFA.

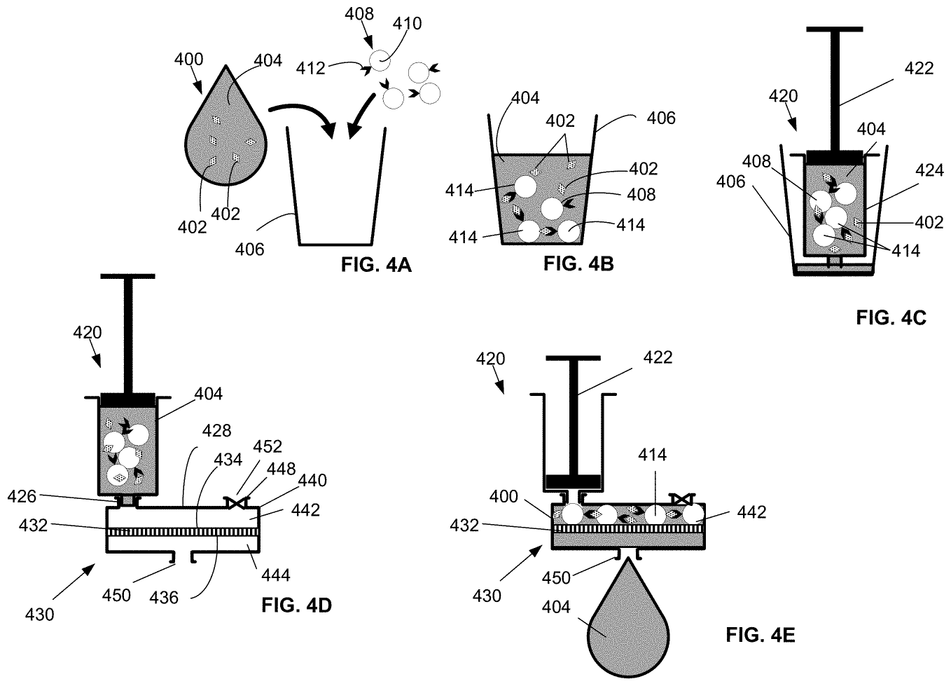

An example system and its use in concentration, washing, and elution steps is depicted in FIGS. 4A-4M. This system is carried out with a luer lock syringe in combination with a small disk filter. In FIG. 4A, sample solution 400 containing analyte 402 in fluid component 404, is placed in container 406, as are functionalized particles 408 made up of particles 410 functionalized with capture ligand 412, and incubated at FIG. 4B, which is generally as described in connection with FIGS. 2A-2C. Fluid component 404, containing functionalized particle-captured analyte complex 414 (as well as any unbound analyte 402 and/or unbound functionalized particles 408) is drawn into syringe 420 from container 406, as shown in FIG. 4C. Syringe 420 includes removable plunger 422 which provides easy access to barrel 424.

A depicted in FIG. 4D, syringe 420 mates with inlet port 426 on filter housing 428 of filtration concentration device 430. Filtration-concentration device 430 includes a filter membrane 432 having a first side 434 and second side 436. Filter membrane 432 has pores small enough to block passage of a functionalized nanoparticle-captured analyte complex 414 from first side 434 to second side 436 but large enough to permit passage of fluid or unbound analyte from first side 434 to second side 436. Filtration-concentration device 430 also includes housing 440, which is configured to contain filter membrane 432. Housing 440 has an upstream chamber 442 in fluid communication with the first side 434 of filter membrane 432 and downstream chamber 444 in fluid communication with second side 436 of filter membrane 432. Filter membrane 432 is designed with an optimum holdup (dead) volume to yield the right sample volume for the downstream assay (e.g. 100 μl). Filter membrane 432 can be formed from various materials, including, for example, at least one of organic membranes made up of one or more polymers, as cellulose, nitrocellulose, cellulose acetate, polysulfone, polyvinylidene fluoride, polyethersulfone, polyethylene sulfone, polyamide, polyethylene teraphthalate, or polytetrafluoroethylene, or glass fiber, having pore size selected to block passage of functionalized particles 408.

Inlet port 426 is in fluid communication with upstream chamber 442 and is adapted to receive fluid sample containing a functionalized nanoparticle-captured analyte complex 414 in a first volume of fluid component 404. Filtration concentration device 430 includes fluid outlet port 450 in fluid communication with downstream chamber 444. Fluid outlet port 450 is configured to permit fluid including a portion of the first volume of fluid to exit filtration concentration device 430. In addition, filtration concentration device 430 includes retentate removal port 448 in communication with upstream chamber 442. Retentate removal port 448 is configured to allow removal of a retentate from the upstream chamber. For example, this can be used to recover functionalized particles 408 for reuse. In an aspect, retentate removal port 448 includes valve 452 which remains closed except when it is desired to remove materials via retentate removal port 448. Fluid outlet port 450 and retentate removal port 448 may include luer lock connectors or other connectors that allow for convenient connection of filtration-concentration device 430 with syringe 420, or with other components. Filter membrane 432 is chemically inert with respect to the functionalized nanoparticle-captured analyte complex 414 and fluid component 404 and exhibits little or no non-specific binding to materials in fluid component 404. Upstream chamber 442 has a volume sufficient to contain a second volume of fluid, wherein the second volume is less than the first volume. The fluidic design of the filtration-concentration device 430, especially the upstream side compartment, may be such that it maximizes the recovery of the concentrated (and optionally also washed) functionalized particle-captured analyte complexes.

As shown in FIG. 4E, plunger 422 of syringe 420 is depressed to drive sample solution 400 into upstream chamber 442, where fluid component 404 passes through filter membrane 432 and exits filtration concentration device 430 via fluid outlet port 450 while functionalized nanoparticle-captured analyte complex 414 is retained in upstream chamber 442.

In FIG. 4F, syringe 420 (or alternatively, another syringe device) containing wash buffer 460 is connected to filtration concentration device 430. Plunger 422 is depressed to push wash buffer 460 through functionalized nanoparticle-captured analyte complex 414 on filter membrane 432, driving residual fluid component 404 through fluid outlet port 450, until as shown in FIG. 4G, functionalized nanoparticle-captured analyte complex 414 on filter membrane 432 is cleaned and surrounded by wash buffer 460. Wash buffer 460 can be, for example, phosphate buffered saline (PBS). As depicted in FIGS. 4H and 4I, a syringe 420 can be used to push air 464 through filtration concentration device 430 to drive remaining wash buffer 460 out of upstream chamber 442 and out of fluid outlet port 450, leaving substantially dry functionalized nanoparticle-captured analyte complex 414 on filter membrane 432 in upstream chamber 442.

In cases where the target analyte needs to be freed from the functionalized particle-captured analyte complexes 414 following concentration and washing as described above, an elution or release buffer can next be pushed through the bed of functionalized particle-captured analyte complexes releasing the analyte. In FIGS. 4J and 4K, syringe 420 is used to push a small volume of elution buffer 466 through filtration concentration device 430. Analyte 402 is eluted from functionalized particles 408, and carried through filter membrane 432 and exits filtration concentration device 430 via fluid outlet port 450, carried in elution buffer 466. Elution buffer 466 with analyte 402 can be applied to a lateral flow assay or other assay for detection, as described elsewhere herein.

FIGS. 4L and 4M illustrate retrieval of functionalized particles 408 for reuse. A first syringe 420 is connected to inlet port 426, and a second syringe 468 is connected to retentate removal port 448. Fluid outlet port 450 is closed, e.g. by closing a valve 470. Suitable carrier fluid (e.g., wash buffer 460) is injected into upstream chamber 442 using first syringe 420, plunger 472 of second syringe 468 is simultaneously withdrawn to draw functionalized particles 408 in wash buffer 460 into second syringe 468, and captured for reuse.

In some aspects, functionalized particles 408 are suitable to be used in a lateral flow assay or other bead-based assay, in that they include a detectable component (e.g., latex beads, colloidal gold particles, other colloidal metals, colloidal carbon, fluorescent or luminescent labels, quantum dots, upconverting phosphores, bioluminescent markers, enzymes, magnetic or paramagnetic particles, dyes, electroactive compounds, or other suitable labels or markers, as described above in connection with FIGS. 3A-3C). Thus it is not necessary to elute analyte 402 off of the functionalized particles. In such applications, functionalized particle-captured analyte complexes 414 can be removed from filtration concentration device 430 following washing, e.g. at FIG. 4G or 4I, and a dual syringe setup like that used in FIGS. 4L and 4M can be used to retrieve functionalized particle-captured analyte complexes 414 in a buffer suitable for application to a lateral flow assay.

Application of functionalized particle-captured analyte complexes 414 directly to a half-strip lateral flow assay device 500 is illustrated in FIGS. 5A-5C. Half-strip lateral flow assay device 500 is a lateral flow assay without the sample pad or conjugate pad or functionalized particles present. For example, functionalized particle-captured analyte complexes 414 (made up of functionalized particle 408 and captured analyte 402) can be removed from separation structure 208, following removal of wash buffer 214, e.g., as depicted in FIG. 2F, or FIG. 4G or 4I. In FIG. 5A, sample 502 containing functionalized particle-captured analyte complexes 414 in carrier fluid 504 (which may be wash buffer or running buffer) is applied at first end 506 of half strip lateral flow assay device 500, and moves via capillary forces to second end 508, in the direction indicated by the heavy black arrow. In this case, half strip lateral flow assay device 500 does not include a conjugate pad containing antibodies specific to analyte 402; instead, functionalized particles 408 in functionalized particle-captured analyte complexes 414 include a detectable component, as noted above. Half strip lateral flow assay device 500 does, however, contain antibodies 318 that are specific to analyte 402, immobilized on the material forming the assay flow path, which may be localized at a test line 316 as depicted in FIG. 5A. In an aspect, half strip lateral flow assay device 500 also includes a control line 320 containing antibodies 322, which are specific to functionalized particles 408.

FIG. 5B depicts half strip lateral flow assay device 500 after sufficient time has passed for carrier fluid 504 to spread to fill first end 506 of lateral flow assay device and travel downstream through lateral flow assay device 500, to the location of flow front 526.

FIG. 5C depicts half strip lateral flow assay device 500 after flow front 526 of carrier fluid 504 has travelled to second end 508 of half strip lateral flow assay device 500. As carrier fluid 504 travels past test line 316, functionalized particle-captured analyte complexes 414 are bound by antibodies 318. Unbound functionalized particles 408 bind to antibodies 322 at control line 320. Functionalized particle-captured analyte complexes 414 captured at test line 316, and functionalized particles 408 at control line 320 both include detectable components that produce a detectable signal that indicates presence of analyte 402 (at test line 316) and presence of properly functioning assay components (at control line 320), respectively.

Sample Filtration Container for Use with Particulate Capture Medium

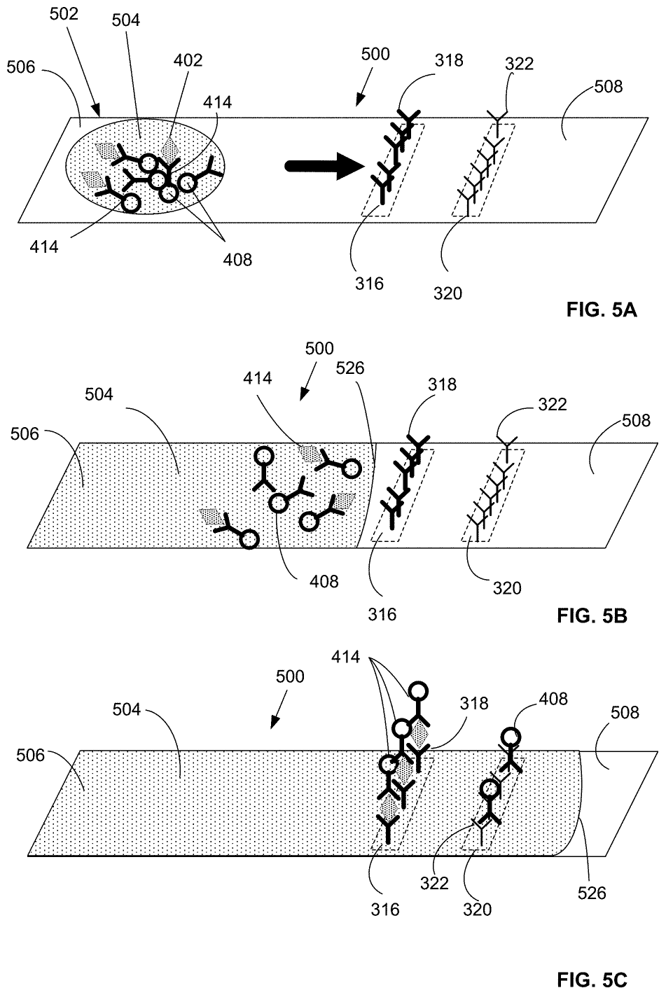

FIGS. 6A-6B depicts a sample filtration container that can be used to concentrate nanoparticles with captured antigen, which can then be transferred to a half strip LFA as illustrated in FIGS. 5A-5C. FIG. 6A depicts a sample filtration container 600 that includes a base 602 defining a bottom of sample filtration container 600, at least one side wall 604. Side wall 604 is contiguous with base 602, and encloses interior 606 of sample filtration container 600. Sample filtration container 600 includes opening 608 at a top 610 of sample filtration container 600. Opening 608 is adapted to receive a sample 614 including a fluid component 616 and a particulate material 618 carried in the fluid component, as depicted in FIG. 6A. Particulate material 618 may include, for example, functionalized particles that can be incubated with an analyte-containing sample fluid to form functionalized particle-captured analyte complexes, previously-formed functionalized particle-captured analyte complexes, or other types of particulate materials, without limitation. Sample filtration container 600 includes divider 620 located within the interior of the sample filtration container, which divides the interior of sample filtration container 600 into an upper portion 622 and lower portion 624. Divider 620 includes a size exclusion filter 626, wherein the size exclusion filter 626 has a first side 628 communicating with upper portion 622 of sample container 600 and a second side 630 communicating with lower portion 624 of sample container 600. Size exclusion filter 626 has a pore size adapted to allow passage of the fluid component 616 of sample 614 while blocking passage of the particulate material 618. In an aspect, size exclusion filter 626 is formed from various materials, including, for example, at least one of organic membranes made up of one or more polymers, as cellulose, nitrocellulose, cellulose acetate, polysulfone, polyvinylidene fluoride, polyethersulfone, polyethylene sulfone, polyamide, polyethylene teraphthalate, or polytetrafluoroethylene, or glass fiber, having pore size selected to block passage of functionalized particles 408. Sample filtration container 600 also includes a capillary medium 632 within lower portion 624 of sample container 600. Capillary medium 632 is adapted to draw fluid component 616 of sample 614 through size exclusion filter 626 from the upper portion 622 to the lower portion 624 of the sample container. Capillary medium 632 may retain the fluid component within the lower portion of the sample container. Capillary medium 632 includes, for example, a cellulosic material, a fiber-based material, a glass fiber, a sponge, a resin, a superabsorbent polymer (e.g., sodium polyacrylate or potassium polyacrylate), or a hydrogel.

In an aspect, a flow control feature 634 is located between upper portion 622 and lower portion 624, either above or below size exclusion filter 626. In FIG. 6A, flow control feature 634 is shown below size exclusion filter 626. In the example of FIG. 6A, flow control feature 634 is a dissolvable sacrificial layer (formed from, e.g., a sugar or pullulan). Alternative, the flow control feature may be a mechanically actuated valve or a mechanism that keeps upper portion 622 and lower portion 624 physically separate and clicks them together on demand. Flow control feature 634 is used when it is desirable to keep upper portion 622 and lower portion 624 physically separate so that fluid component 616 is retained in upper portion 622 long enough to allow incubation for analyte capture to occur. In the event that capture incubation occurs rapidly, or if it the incubation step is performed in another container and already-formed capture bead-analyte complex is poured into upper portion 622, then flow control feature 634 may be omitted.

As shown in FIG. 6B, after flow control feature 634 has dissolved (or been opened in some other manner), fluid component 616 flows through size exclusion filter 626 into lower portion 624, drawn by capillary forces exerted by capillary medium 632, while particulate material 618 remains in upper portion 622, concentrated into a small residual volume of fluid component 616. If desired, a wash buffer can be added to sample filtration container 600. The wash buffer will similarly be drawn into the capillary medium 632 leaving concentrated, cleaned particles in upper portion 622. Particles can be manually removed for further processing, e.g. for elution of analyte.

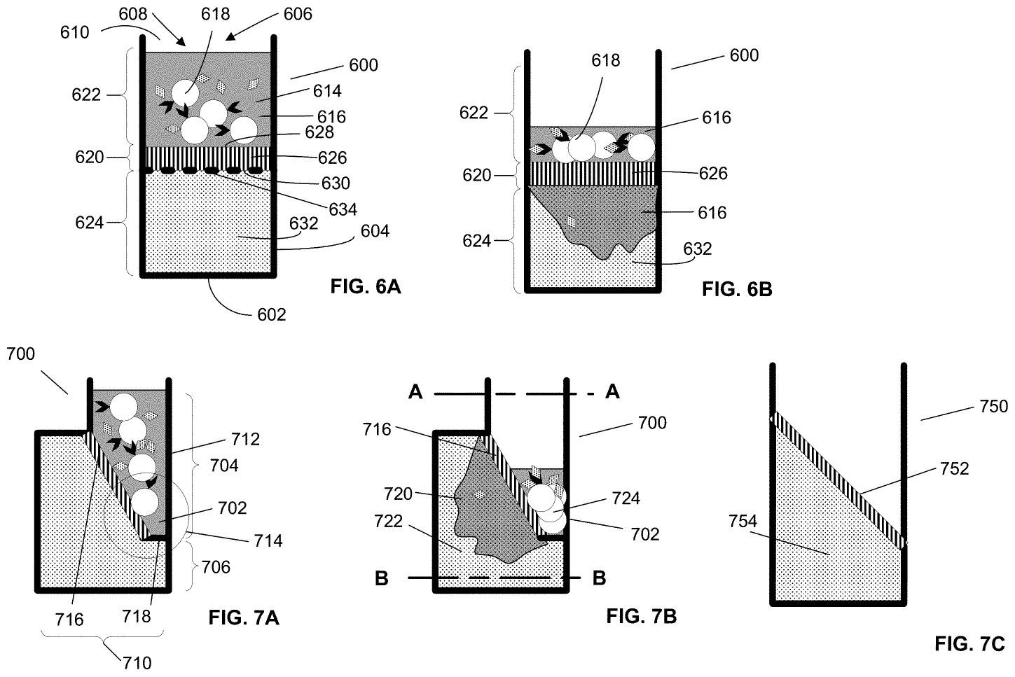

As depicted in FIGS. 7A and 7B, in an aspect a sample filtration container 700, includes sample collection region 702 located at a bottom of the upper portion 704 of the sample filtration container 700, where sample collection region 702 has a reduced cross-sectional area relative to the upper portion 704 as a whole. Upper portion 704 is separated from lower portion 706 by divider 710, which in this case is substantially planar and oriented at an oblique angle relative to side wall 712. Sample collection region 702 is bounded by the at least one side wall and the divider at a location 714 (circled in FIG. 7A) at which the divider 710 forms an acute angle with side wall 712. It should be noted that as shown in FIGS. 7A and 7B, divider 710 is substantially but not absolutely planar; it is formed by the combination of size exclusion filter 716 and wall extension 718, which in this example are not co-planar. As shown in FIG. 7B, fluid component 720 is drawn across size exclusion filter 716 into capillary medium 722, while particulate material 724 collects in sample collection region 702. Particulate material 724 can be removed, for example, by pipetting. FIG. 7C depicts an alternative sample filtration container 750 in which size exclusion filter 752 functions as a divider. Capillary medium is indicated at 754. Sample filtration containers 600, 700 and 750 depicted in FIGS. 6A-6B and 7A-7C are depicted only in cross section. Containers 600 and 750 may take the form of a cylinder or rectangular prism. Container 700 may have a rectangular, circular, or ovoid cross-section at section lines A-A or B-B. The particular dimensions of the containers can be selected for ease of manufacture, storage, or handling, and/or to provide desired absolute and relative volumes of the upper and lower portions of the containers. For example, container 700 provides for a relatively larger volume of capillary medium 722 in lower portion 706 compared to the volume of fluid that can be contained in upper portion 704. This may be useful, for example, if capillary medium 722 is intended to have the capacity to absorb fluid components of both sample and wash buffer.

In other aspects, not shown, a divider may include a shaped depression, with the sample collection region located at the bottom of the shaped depression. For example, the divider may have an inverted conical shape, in which vertex of the cone forms the depression in which particulate material collects. Various other configurations of divider 710 can be utilized to form a reduced cross-section sample collection region. In some aspects, the divider can be configured to be removed from the sample filtration container, and can serve as a transfer device for the concentrated beads.

### Method of Using Sample Filtration Container

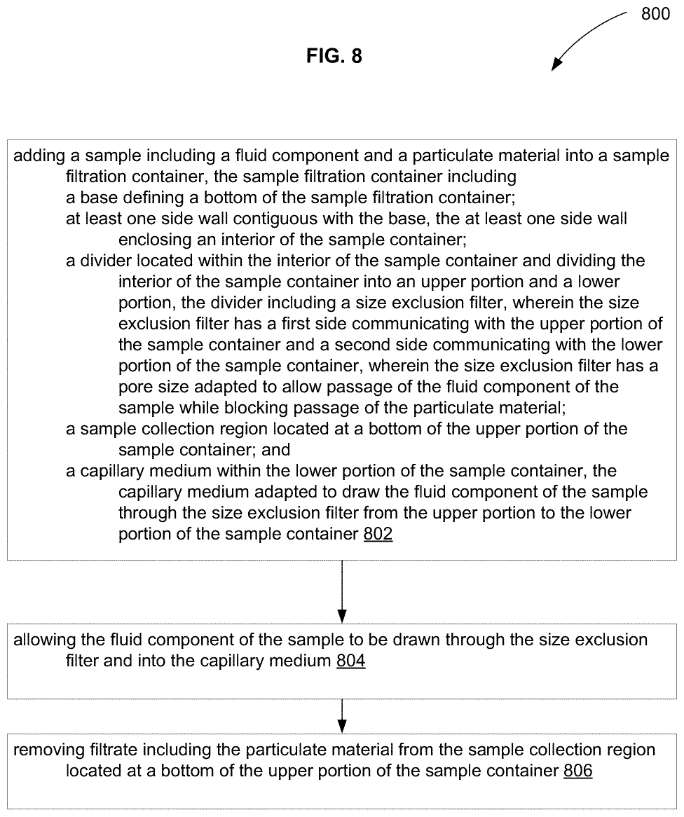

FIG. 8 depicts a method of using a sample filtration container of the type depicted in FIGS. 6A-6B and 7A-7C. In an aspect, a method 800 of filtering a sample includes adding a sample including a fluid component and a particulate material into a sample filtration container, the sample filtration container including a base defining a bottom of the sample filtration container; at least one side wall contiguous with the base, the at least one side wall enclosing an interior of the sample container; a divider located within the interior of the sample container and dividing the interior of the sample container into an upper portion and a lower portion, the divider including a size exclusion filter, wherein the size exclusion filter has a first side communicating with the upper portion of the sample container and a second side communicating with the lower portion of the sample container, wherein the size exclusion filter has a pore size adapted to allow passage of the fluid component of the sample while blocking passage of the particulate material; a sample collection region located at a bottom of the upper portion of the sample container; and a capillary medium within the lower portion of the sample container, the capillary medium adapted to draw the fluid component of the sample through the size exclusion filter from the upper portion to the lower portion of the sample container, as indicated at 802; allowing the fluid component of the sample to be drawn through the size exclusion filter and into the capillary medium, as indicated at 804; and removing filtrate including the particulate material from the sample collection region located at a bottom of the upper portion of the sample container, as indicated at 806.

### Methods of Filtering Particulates Applied to LFA

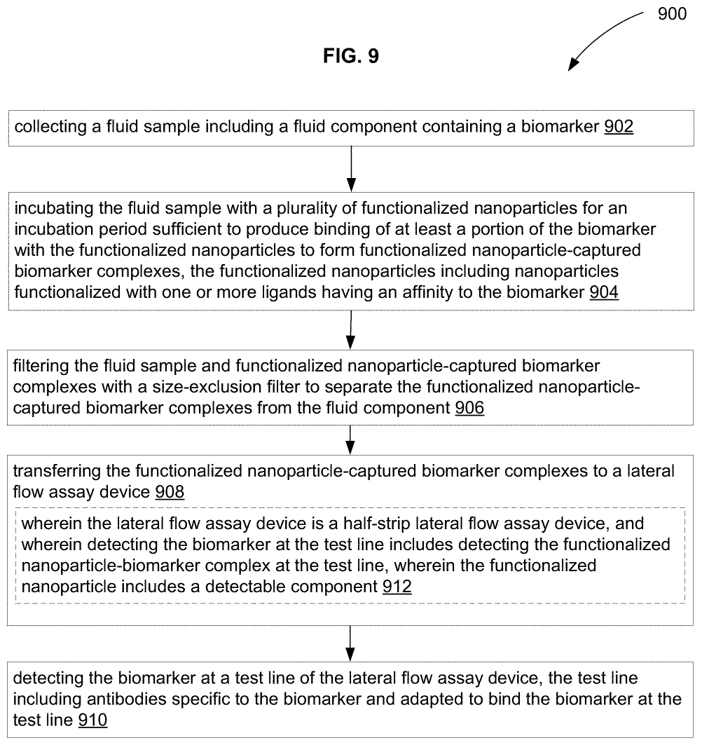

In an aspect, after analyte of interest is capture on a functionalized nanoparticle, functionalized nanoparticle-captured analyte complexes are applied directly to an LFA, rather than first eluting the analyte from the functionalized nanoparticle and applying a solution containing the eluted analyte to the LFA. FIG. 9 is a flow diagram of such a method. As shown in FIG. 9, a method 900 of detecting a biomarker of interest from a fluid sample includes collecting a fluid sample including a fluid component containing a biomarker, as indicated at 902; incubating the fluid sample with a plurality of functionalized nanoparticles for an incubation period sufficient to produce binding of at least a portion of the biomarker with the functionalized nanoparticles to form functionalized nanoparticle-captured biomarker complexes, the functionalized nanoparticles including nanoparticles functionalized with one or more ligands having an affinity to the biomarker, as indicated at 904; filtering the fluid sample and functionalized nanoparticle-captured biomarker complexes with a size-exclusion filter to separate the functionalized nanoparticle-captured biomarker complexes from the fluid component, as indicated at 906; transferring the functionalized nanoparticle-captured biomarker complexes to a lateral flow assay device, as indicated at 908; and detecting the biomarker at a test line of the lateral flow assay device, the test line including antibodies specific to the biomarker and adapted to bind the biomarker at the test line, as indicated at 910. In an aspect, method 900 includes washing the functionalized nanoparticle-captured biomarker complexes on the size-exclusion filter with a wash buffer prior to transferring the functionalized nanoparticle-captured biomarker complexes to the lateral flow assay device, for example as described generally in connection with FIGS. 2E and 2F.

In aspect, the lateral flow assay device is a half-strip lateral flow assay device, and wherein detecting the biomarker at the test line includes detecting the functionalized nanoparticle-biomarker complex at the test line, wherein the functionalized nanoparticle includes a detectable component, as indicated at 912. This approach is illustrated in FIGS. 5A-5C). In this approach, the same nanoparticles used for capture-concentration of the biomarker are also used for detection on the lateral flow assay.

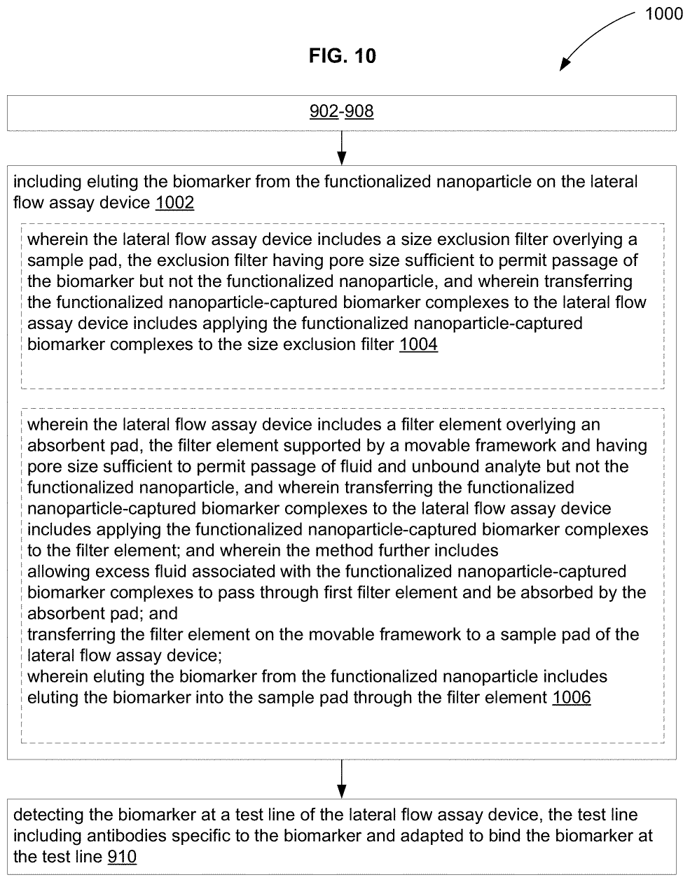

In another aspect, functionalized nanoparticle-biomarker complex is applied to an LFA and elution of analyte is performed on the LFA. FIG. 10 depicts such an approach, which is a variant of the general method shown in FIG. 9. Method steps 902-908 are as discussed in connection with FIG. 9. Method 1000 includes eluting the biomarker from the functionalized nanoparticle on the lateral flow assay device, as indicated at 1002. In general, this approach involves filtration of functionalized nanoparticle-biomarker complex using a filter attached to or built into the LFA cassette, and elution of analyte from nanoparticles performed on the LFA. For example, following elution of analyte on the LFA, the method includes exposing the eluted biomarker to an antibody specific to the biomarker on the lateral flow assay device, wherein the antibody specific to the biomarker is conjugated to a detectable component, and wherein detecting the biomarker at a test line includes detecting the detectable component.

In an aspect, the lateral flow assay device includes a size exclusion filter overlying a sample pad, the exclusion filter having pore size sufficient to permit passage of the biomarker but not the functionalized nanoparticle, and wherein transferring the functionalized nanoparticle-captured biomarker complexes to the lateral flow assay device includes applying the functionalized nanoparticle-captured biomarker complexes to the size exclusion filter, as indicated at 1004.

In another aspect of method 1000, the lateral flow assay device includes a filter element overlying an absorbent pad, the filter element supported by a movable framework and having pore size sufficient to permit passage of fluid and unbound analyte but not the functionalized nanoparticle, and transferring the functionalized nanoparticle-captured biomarker complexes to the lateral flow assay device includes applying the functionalized nanoparticle-captured biomarker complexes to the filter element; and wherein the method further includes allowing excess fluid associated with the functionalized nanoparticle-captured biomarker complexes to pass through first filter element and be absorbed by the absorbent pad, and transferring the filter element on the movable framework to a sample pad of the lateral flow assay device; wherein eluting the biomarker from the functionalized nanoparticle includes eluting the biomarker into the sample pad through the filter element, as indicated at 1006.

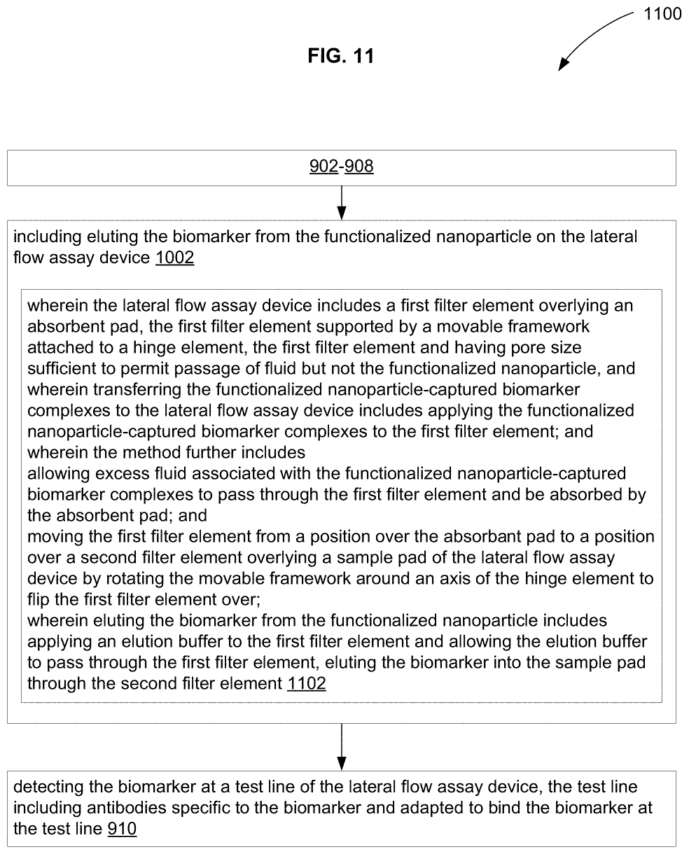

FIG. 11 depicts additional aspects of a related method 1100 (in which steps 902-908 and 1002 are as discussed herein above). In an aspect of method 1100, the lateral flow assay device includes a first filter element overlying an absorbent pad, the first filter element supported by a movable framework attached to a hinge element, the first filter element and having pore size sufficient to permit passage of fluid but not the functionalized nanoparticle, and wherein transferring the functionalized nanoparticle-captured biomarker complexes to the lateral flow assay device includes applying the functionalized nanoparticle-captured biomarker complexes to the first filter element; and wherein the method further includes allowing excess fluid associated with the functionalized nanoparticle-captured biomarker complexes to pass through the first filter element and be absorbed by the absorbent pad, and moving the first filter element from a position over the absorbent pad to a position over a second filter element overlying a sample pad of the lateral flow assay device by rotating the movable framework around an axis of the hinge element to flip the first filter element over, wherein eluting the biomarker from the functionalized nanoparticle includes applying an elution buffer to the first filter element and allowing the elution buffer to pass through the first filter element, eluting the biomarker into the sample pad through the second filter element, as indicated at 1102.

In various aspects, the eluent flows from the filter to the LFA via a flow path that is connected manually (e.g., by sliding, hinging, clicking, depressing, pull tab) or automatically, e.g. through swelling of a wet absorbing material, removal of a dissolvable flow barrier (complex sugar or such), or opening of a surface tension valve.

LFA with Particle Filter

As discussed herein above, in some cases the concentrated functionalized particle-captured analyte complex are placed directly onto a specially designed LFA having a coarse membrane on the sample pad to retain the beads but permit passage of the liberated analyte.

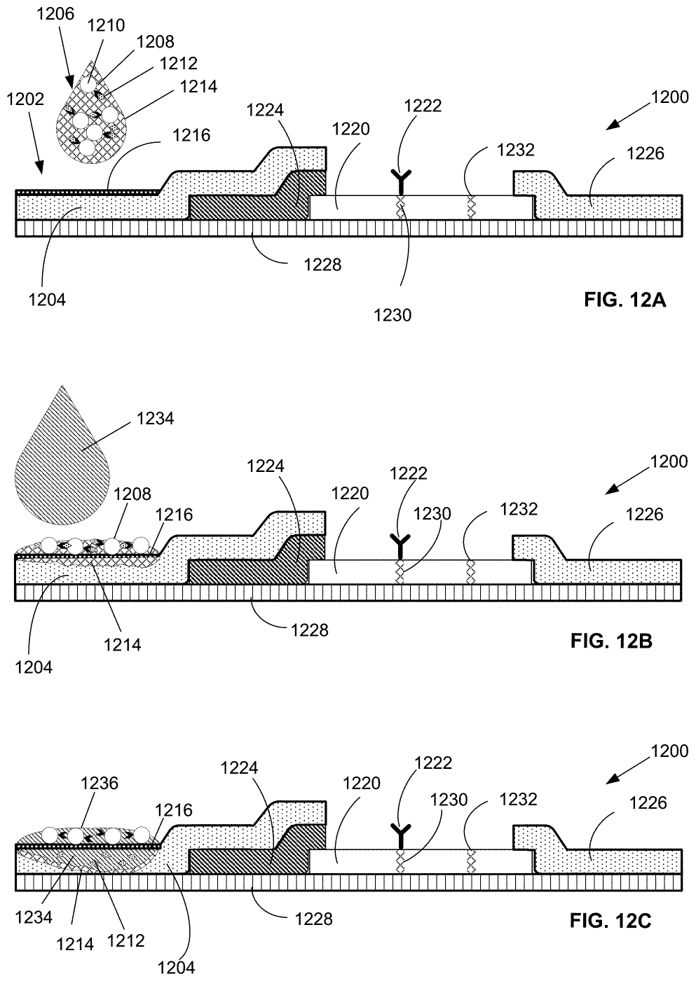

FIGS. 12A-12C depict an LFA which includes a concentration/separation portion adapted for receiving functionalized particle-captured analyte complex, e.g. from a sample filtration container as depicted in FIGS. 6A and 6B and FIGS. 7A-7C. Lateral flow assay device 1200 includes a loading region 1202 including sample pad 1204. Loading region 1202 is adapted to receive a fluid 1206 containing a functionalized nanoparticle-captured analyte complex 1208 including one or more functionalized nanoparticle 1210 and an analyte of interest 1212 in a carrier fluid 1214.

Lateral flow assay device 1200 includes filter element 1216 overlying sample pad 1204, wherein the filter element 1216 includes pores small enough to block passage of the functionalized nanoparticle 1210 through the filter element 1216 but large enough to permit passage of the carrier fluid 1214 and unbound analyte of interest 1212 through filter element 1216 to sample pad 1204. In an aspect, filter element 1216 is formed from various materials, including, for example, at least one of organic membranes made up of one or more polymers, as cellulose, nitrocellulose, cellulose acetate, polysulfone, polyvinylidene fluoride, polyethersulfone, polyethylene sulfone, polyamide, polyethylene teraphthalate, or polytetrafluoroethylene, or glass fiber. In addition, lateral flow assay device 1200 includes lateral flow membrane 1220 downstream of sample pad 1204 and including one or more capture components 1222 adapted to capture analyte of interest 1212. In an aspect, lateral flow assay device 1200 also includes conjugate pad 1224, wick 1226, and backing 1228. In an aspect, lateral flow membrane 1220 includes test line 1230 including the one or more capture components 1222. In some aspects, lateral flow membrane 1220 also includes control line 1232.

As shown in FIG. 12A, functionalized particle-captured analyte complex 1208 in carrier fluid 1214 is applied to filter element 1216. Carrier fluid 1214 may be a wash buffer from a previous processing step, for example. Carrier fluid 1214 passes through filter element 1216 and is absorbed into sample pad 1204, while functionalized particle-captured analyte complex 1208 remains on top of filter 1216. In FIG. 12B, elution buffer 1224 is applied to functionalized particle-captured analyte complex 1208 on filter element 1216. As shown in FIG. 12C, analyte 1212 is eluted from functionalized particle 1236 and carried into sample pad 1204 by elution buffer 1234. Analyte 1212 in elution buffer 1224 travels through conjugate pad 1224 and lateral flow membrane 1220 where it is captured at test line 1230. Elution buffer 1234 (or other running buffer) and any uncaptured analyte 1212 eventually travel toward and into wick 1226. Sample pad 1204 functions to control the rate at which sample fluid enters conjugate pad 1224. Sample pad 1204 may contain proteins, detergents, viscosity enhancers, buffers, salts, or other materials that improve the properties of the sample fluid. In an aspect, the one or more capture components 1222 include one or more capture component adapted to capture TB LAM. In an aspect, the one or more capture components 1222 include one or more antibody adapted to capture TB LAM. In an aspect, the sample pad 1204 is capable of absorbing about 5 ml. In various aspects, the sample pad 1204 includes at least one of cellulose, glass fiber, cotton, rayon, a woven mesh, and a synthetic non-woven material (see, e.g., Brendan O'Farrell, “Chapter 1. Evolution in Lateral Flow-Based Immunoassay Systems”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500ENOOEM Rev. C 12/13, © 2013, EMD Millipore Corporation, Billerica, Mass., both of which are incorporated herein by reference). For example, suitable materials include, but are not limited to, SureWick® glass fiber pads and cellulose pads from EMD Millipore, Billerica, Mass. and CF1 to CF7 100% cotton linter pads from GE Healthcare Biosciences, Pittsburgh, Pa.). In an aspect, the filter element 1216 has a pore size of between about 0.1 μm and about 0.4 μm. Choice of a suitable pore size is dependent on the size of functionalized nanoparticle 1210. In an aspect, the filter element 1216 is formed of a chemically inert material having minimal nonspecific binding to components of the fluid. In an aspect, the filter element 1216 is formed of a mildly hydrophilic material. In an aspect, conjugate pad 1224 contains dried conjugate (e.g., conjugate antibodies and detectable component as discussed in connection with FIGS. 3A-3C). In various aspects, conjugate pad 1224 is formed from glass fiber, polyesters, cotton, or rayon, e.g. as discussed in “Rapid Lateral Flow Test Strips: Considerations for Product Development,” Lit. No. TB500ENOOEM Rev. C 12/13, © 2013, EMD Millipore Corporation, Billerica, Mass., which is incorporated herein by reference. In an aspect, lateral flow membrane 1220 is a porous membrane with well-defined capillary flow properties that provides a uniform and controlled flow of fluid to test line 1230 and control line 1232. In various aspects, lateral flow membrane materials include nitrocellulose, polyvinylidene fluoride, charge-modified nylon, polyether sulfone, nitrocellulose acetate, glass fiber, cellulose, paper, silica, a porous synthetic polymer, polyester, nylon, cotton, a sintered material, a woven material, or a non-woven material. Lateral flow membrane materials may be treated with surfactant to improve the wettability of the membrane. (See, e.g. Michael A. Mansfield, “Chapter 6. Nitrocellulose Membranes for Lateral Flow Immunoassays: A Technical Treatise”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3; E. J. Flynn, J. Arndt, L. Brothier, and M. A. Morris (2013), “Control of pore structure formation in cellulose nitrate polymer membranes,” Advances in Chem. Science., Vol. 2, Issue 2, June 203, pp. 9-18; and “Rapid Lateral Flow Test Strips: Considerations for Product Development,” Lit. No. TB500EN00EM Rev. C 12/13, © 2013, EMD Millipore Corporation, Billerica, Mass., each of which is incorporated herein by reference, for discussion of lateral flow membrane and conjugate pad materials).

In an aspect, test line 1230 contains immobilized antibodies specific to the analyte of interest, bound irreversibly to lateral flow membrane 1220, and a control line 1232 contains immobilized antibodies specific to conjugate antibodies. Wick 1226 (an absorbent pad) is located at the downstream end of lateral flow membrane 1220. In various aspects, wick 1226 includes at least one of cellulose, high-density cellulose, glass, polyester, nylon, cotton, mono-component fiber, or bi-component fiber (see, e.g., Brendan O'Farrell, “Chapter 1. Evolution in Lateral Flow-Based Immunoassay Systems”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500ENOOEM Rev. C 12/13, © 2013, EMD Millipore Corporation, Billerica, Mass., both of which are incorporated herein by reference).

In an aspect, sample pad 1204, conjugate pad 1224, lateral flow membrane 1220, and wick 1226 are formed on backing 1228. In various aspects, backing 1228 includes a non-porous plastic film or card, including one or more of polystyrene, vinyl (poly vinyl chloride or PVC), or polyester. In various aspects, backing 1228 includes an adhesive, which may be covered by a release liner. Thickness of the backing may be for example 0.0005 to 0.015 inches, with thicker materials typically used for stand-alone test strips, while thinner materials may be used in a holder or housing (see, e.g. Jennifer S. Ponti, “Chapter 3. Material Platform for the Assembly of Lateral Flow Immunoassay Test Strips”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500ENOOEM Rev. C 12/13, © 2013, EMD Millipore Corporation, Billerica, Mass., both of which are incorporated herein by reference). Although it is typical that lateral flow assay devices are formed on a backing, in some cases the materials forming the lateral flow assay device are sufficiently self-supporting that the backing can be omitted. Unless otherwise noted, materials and construction of other lateral flow assay devices described herein are similar to the materials and construction discussed of the device described and depicted in connection with FIGS. 12A-12C.

LFA with Translating Particle Transfer Mechanism

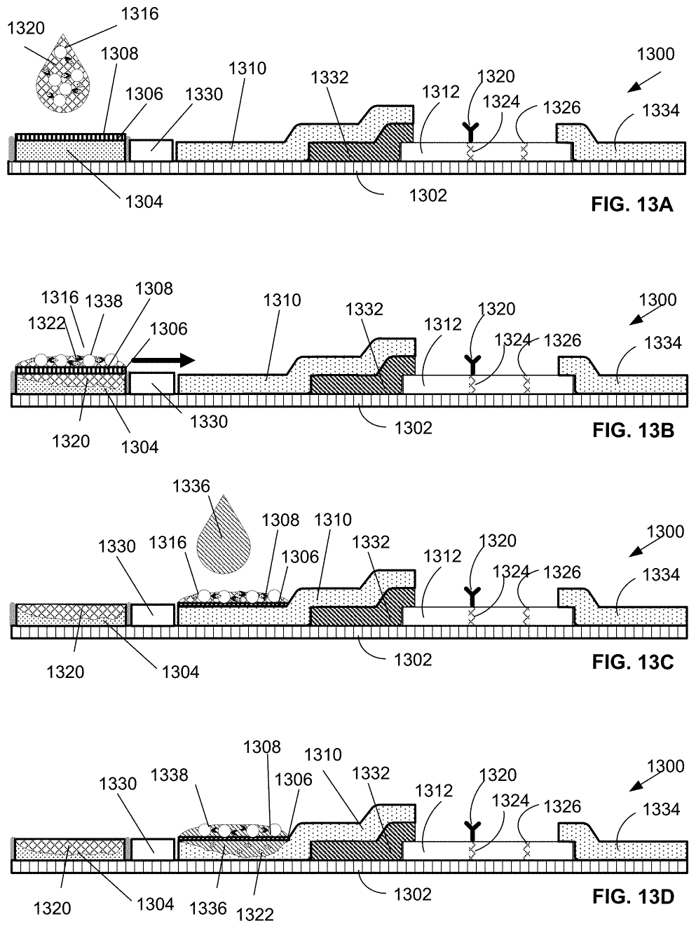

FIGS. 13A-13D depict a lateral flow assay device 1300 that includes a concentration/separation device and a particle transfer mechanism. Lateral flow assay device 1300 includes a support layer 1302 (e.g., similar to backing 1228 in FIG. 12A-12C), an absorbent pad 1304 disposed on support layer 1302, a movable framework 1306 configured to fit closely and removably over the absorbent pad 1304, a first filter element 1308 supported by movable framework 1306, sample pad 1310, and lateral flow membrane 1312. First filter element 1308 is configured for fluid communication with the absorbent pad 1304 through one or more apertures in movable framework 1306, wherein the first filter element 1308 includes pores small enough to block passage of a functionalized nanoparticle-captured analyte complex 1316 through the first filter element 1308 but large enough to permit passage of a carrier fluid 1320 through first filter element 1308 to absorbent pad 1304. Sample pad 1310 is supported by support layer 1302 and configured so that movable framework 1306 can be fit closely over sample pad 1310. Lateral flow membrane 1312 is located downstream of sample pad 1310 and includes one or more capture components 1320 specific to analyte of interest 1322. In an aspect, the first filter element 1308 includes pores large enough to permit passage of unbound analyte 1322 through first filter element 1308 to absorbent pad 1304. Pore size thus depends on the size of the functionalized nanoparticles to be captured. In an aspect, the first filter element 1308 has a pore size between about of about 0.1 μm and about 0.4 μm.

In an aspect, first filter element 1308 is formed of a chemically inert material having minimal nonspecific binding to components of the fluid sample. In an aspect, the first filter element 1308 is formed of a mildly hydrophilic material. Possible materials for first filter element 1308 includes, for example, at least one of organic membranes made up of one or more polymers, as cellulose, nitrocellulose, cellulose acetate, polysulfone, polyvinylidene fluoride, polyethersulfone, polyethylene sulfone, polyamide, polyethylene teraphthalate, or polytetrafluoroethylene, or glass fiber. In an aspect, one or more capture components 1320 include one or more capture component adapted to capture TB LAM, e.g. one or more antibody adapted to capture TB LAM. In an aspect, lateral flow membrane 1312 includes a test line 1324 including the one or more capture components 1320. Lateral flow membrane 1312 may also include control line 1326. In an aspect, movable framework 1306 is configured to fit over sample pad 1310 with the first filter element 1308 in fluid communication with sample pad 1310 through one or more apertures in the movable framework. In an aspect, LFA device 1300 also includes conjugate pad 1332 between sample pad 1310 and lateral flow membrane 1312, and wick 1334 downstream of lateral flow membrane 1312.

In FIG. 13A, movable framework 1306 carrying first filter element 1308 is positioned over absorbent pad 1304. Functionalized nanoparticle-captured analyte complex 1316 (made up of analyte of interest 1322 and functionalized nanoparticles 1338) in carrier fluid 1320 is applied to first filter element 1308. As depicted in FIG. 13B, carrier fluid 1320 travels through first filter element 1308 and into absorbent pad 1304, while functionalized nanoparticle-captured analyte complex 1316 remains on top of first filter element 1308. Divider 1330 is a region of non-fluid-conductive material between absorbent pad 1304 and sample pad 1310, which prevents fluid from traveling from absorbent pad 1304 to sample pad 1310, and in an aspect also supports movable framework 1306 as it is moved from absorbent pad 1304 to sample pad 1310. As indicated by the black arrow in FIG. 13B, movable framework 1306 can be moved by translation until it rests over sample pad 1310, as depicted in FIG. 13C. This can be accomplished by sliding movable framework 1306, although alternatively, movable framework 1306 could be picked up and then placed on sample pad 1310. Elution buffer 1336 is applied to functionalized nanoparticle-captured analyte complex 1316 on first filter element 1308. As shown in FIG. 13D, analyte of interest 1322 is eluted and travels through first filter element 1308, through openings in movable framework 1306, and into sample pad 1310 in elution buffer 1336, while functionalized nanoparticles 1338 remain on top of first filter element 1308. Analyte of interest 1322 travels through conjugate pad 1332 and lateral flow membrane 1312 to test line 1324, where it is detected by capture components 1320, e.g. as described elsewhere herein. In an aspect, elution buffer continues through lateral flow membrane 1312 to wick 1334, where it is absorbed.

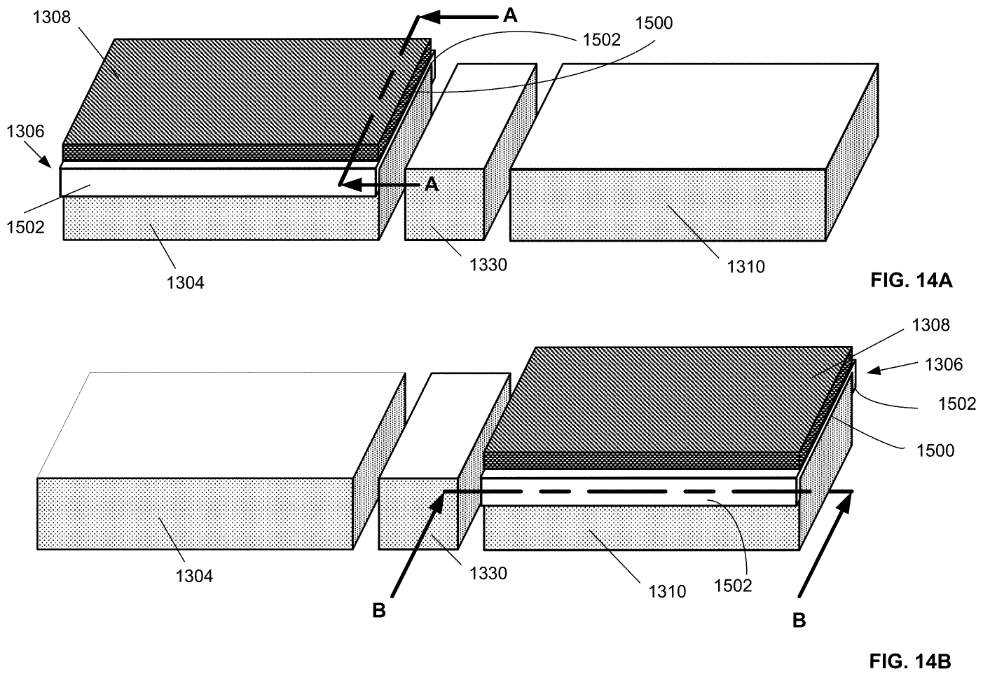

FIGS. 14A and 14B illustrates components of the device of FIGS. 13A-13D in greater detail. FIG. 14A depicts movable framework 1306 carrying first filter element 1308 positioned over absorbent pad 1304, also showing divider 1330 and sample pad 1310. It can be seen that movable framework 1306 is configured to fit closely over absorbent pad 1304, in that top 1500 of movable framework 1306 is slightly wider than absorbent pad 1304, and sides 1502 of movable framework 1306 extend downward from top 1500 adjacent the sides of absorbent pad 1304 to hold movable framework 1306 in position over absorbent pad 1304. Sample pad 1310 has a width substantially the same as that of absorbent pad 1304, such that top 1500 of movable framework 1306 is slightly wider than sample pad 1310, and sides 1502 of movable framework 1306 extend downward from top 1500 adjacent the sides of sample pad 1310 to hold movable framework 1306 in position over sample pad 1310, as depicted in FIG. 14B.

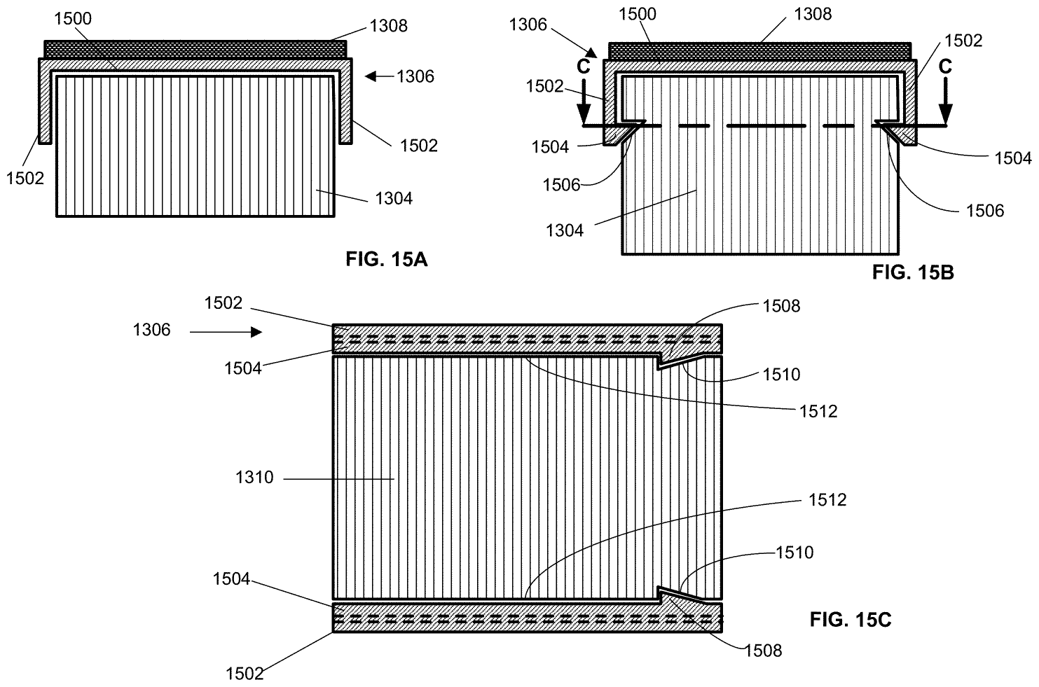

FIG. 15A is a cross-sectional view of first filter element 1308, movable framework 1306, and absorbent pad 1304 taken at section line A-A in FIG. 14A, illustrating the fit of top 1500 and sides 1502 with regard to absorbent pad 1304. FIG. 15B is a cross-sectional view of a related embodiment, in which sides 1502 of movable framework 1306 include elongated projections 1504 which extend into grooves 106 in absorbent pad 1304 (or alternatively, in a rigid housing containing absorbent pad 1304, not shown), allowing movable framework to be slid with respect to absorbent pad 1304 without being lifted. FIG. 15C is a cross-sectional view, taken at section line C-C in FIG. 15B (at a location like that of section line B-B in FIG. 14B) of features that allow movable framework 1306 to be secured in place once it has been slid into the proper position over sample pad 1310. In FIG. 15C, sides 1502 of movable framework 1306 include elongated projections 1504, as depicted in FIG. 15B. In addition, each elongated projection 1504 also includes an angled tooth 1508, which fits into detent 1510 to lock movable framework in place with respect to sample pad 1310 (or a rigid housing containing sample pad 1310). In an aspect, side 1502 flexes outward slightly to allow angled tooth 1508 to slide along side 1512 of sample pad 1310, and snap inward into place when detent 1510 is reached.

LFA with Hinged Particle Transfer Mechanism

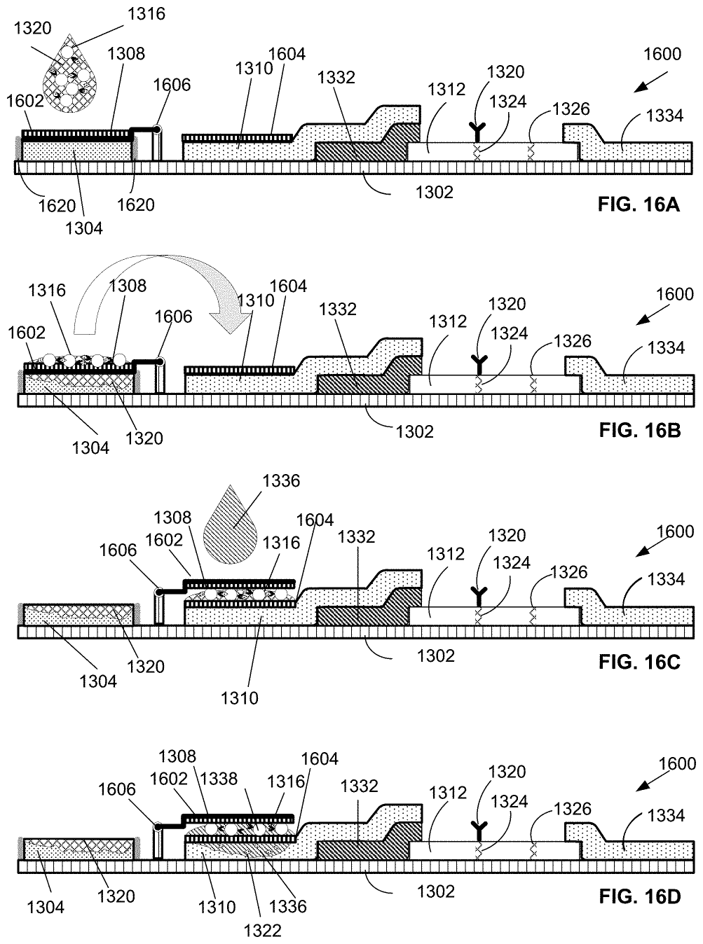

FIGS. 16A-16D depict a lateral flow assay device 1600 similar to that depicted in FIGS. 13A-13D, but having an alternative mechanism for transferring functionalized nanoparticle-captured analyte complex 1316 from absorbent pad 1304 to sample pad 1310. Lateral flow assay device 1600 includes support layer 1302, absorbent pad 1304, first filter element 1308, sample pad 1310, lateral flow membrane 1312, capture components 1320, test line 1324, control line 1326, conjugate pad 1332, and wick 1334 which are as described above in connection with FIGS. 13A-13D. Housing 1620 around absorbent pad 1304 is also depicted. First filter element 1308 is supported by a movable framework 1602, which in this case is hinged and flipped over rather than translated in order to move first filter element 1308 to sample pad 1310. As depicted in FIG. 16A, lateral flow assay device 1600 includes a second filter element 1604 located over the sample pad 1310. Second filter element 1604 is similar to first filter element 1308, in that it includes pores small enough to block passage of the functionalized nanoparticle-captured analyte complex 1316, as well as functionalized nanoparticle without captured analyte, through second filter element 1604 but large enough to permit passage of a second carrier fluid and unbound analyte through the second filter element 1604 to sample pad 1310. First filter element 1308 and second filter element 1604 can be formed from various materials, including, for example, at least one of organic membranes made up of one or more polymers, as cellulose, nitrocellulose, cellulose acetate, polysulfone, polyvinylidene fluoride, polyethersulfone, polyethylene sulfone, polyamide, polyethylene teraphthalate, or polytetrafluoroethylene, or glass fiber. Lateral flow assay device 1600 includes hinge element 1606 disposed on support layer 1302 between absorbent pad 1304 and sample pad 1310 and attached to moveable framework 1602 such that moveable framework 1602 can be rotated around an axis of the hinge element 1606 to flip first filter element 1308 over to move first filter element 1308 from a position over the absorbent pad 1304 to a position over second filter element 1604, so that first filter element 1308 is in fluid communication with sample pad 1310 through the second filter element 1604.

FIG. 16A depicts functionalized nanoparticle-captured analyte complex 1316 in carrier fluid 1320 being applied to first filter element 1308. FIG. 16B depicts functionalized nanoparticle-captured analyte complex 1316 on top of first filter element 1308, while carrier fluid 1320 has traveled through first filter element 1308 and into absorbent pad 1304. Movable framework 1602 is rotated around the axis of hinge element 1606 to flip first filter element 1308 over to move first filter element 1308 from a position over the absorbent pad 1304 to a position over second filter element 1604, in the direction indicated by the curving arrow. FIG. 16C shows movable framework 1602 and first filter element 1308 positioned over second filter element 1604, with functionalized nanoparticle-captured analyte complex 1316 located between first filter element 1308 and second filter element 1604. As depicted in FIG. 16C, elution buffer 1336 is applied, and passes through movable framework 1602, first filter element 1308, second filter element 1604, and into sample pad 1310. As depicted in FIG. 16D, elution buffer 1336 carries analyte 1322 with it into sample pad 1310 but leaving functionalized nanoparticles 1338 on top of second filter element 1604. Analyte 1322 moves through sample pad 1310, conjugate pad 1332, and lateral flow membrane 1312 for detection at test line 1324, as described above. Second filter element 1604 may be similar to or the same as first filter element 1308, e.g. formed of the same types of materials and having similar pore size. In an aspect, second filter element 1604 is formed of a chemically inert material having minimal nonspecific binding to components of the fluid sample. In an aspect, second filter element 1604 is formed of a mildly hydrophilic material.

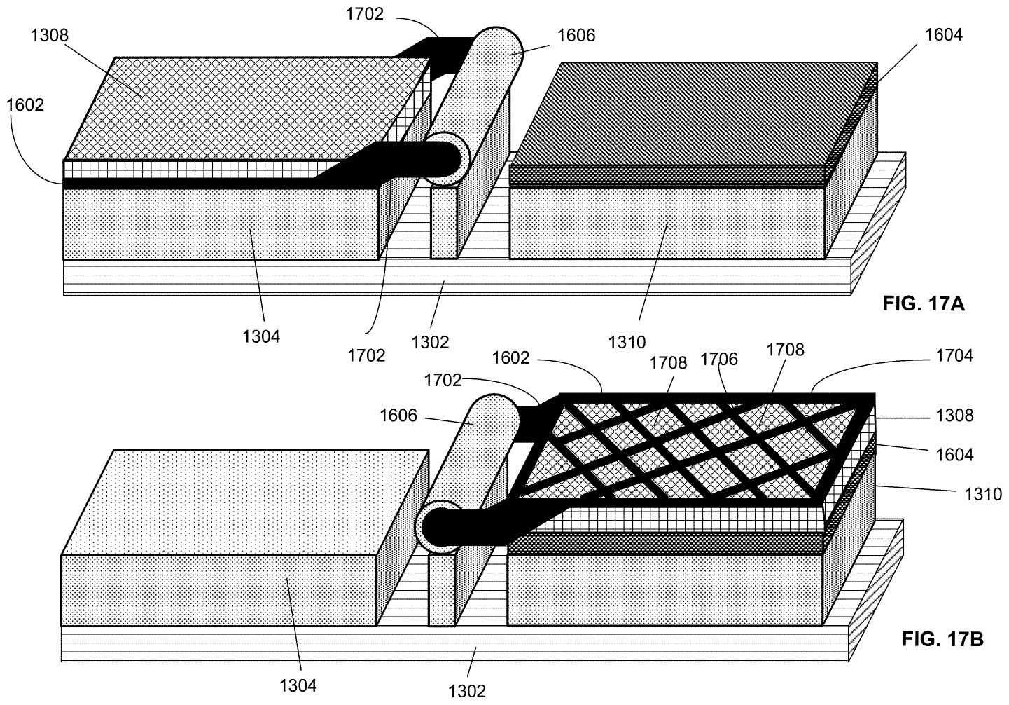

FIGS. 17A-17B illustrate in greater detail the hinging transfer mechanism described in connection with FIGS. 16A-16D. Absorbent pad 1304, and sample pad 1310 are carried on support layer 1302. Movable framework 1602 is connected to hinge element 1606 by arms 1702. FIG. 17A shows first filter element 1308 supported on movable framework 1602. In FIG. 17B, movable framework 1602 has been rotated around hinge element 1606 to flip first filter element 1308 onto second filter element 1604, which is positioned over sample pad 1310. As can be seen movable framework 1602 includes frame 1704 and grid 1706 which supports first filter element 1308 and includes apertures 1708 that allow for passage of fluid.

### Analyte Capture Using Stationary Phase Medium

As discussed above, one approach for capturing and concentrating analytes is to us a functionalized stationary substrate rather than functionalized particles. In this approach, a capture ligand is immobilized in or on a stationary phase that resides in a fixed part of a device and the sample containing analyte is flowed through the stationary phase one or more times. Depending on interplay of kinetics and device design, passing sample through the stationary phase once may be sufficient to capture the analyte; alternatively, it may be preferable to pass sample containing analyte through the stationary phase multiple times, in some cases with repeated back and forth flow, to provide sufficient opportunity for the analyte to be captured. Similarly, wash buffer can be flowed through one or multiple times as needed to reduce background. Eventually, a small volume of release buffer can be added to release analyte in concentrated form. Several examples using a stationary phase medium are provided below.

Capture Concentration with Functionalized Membrane

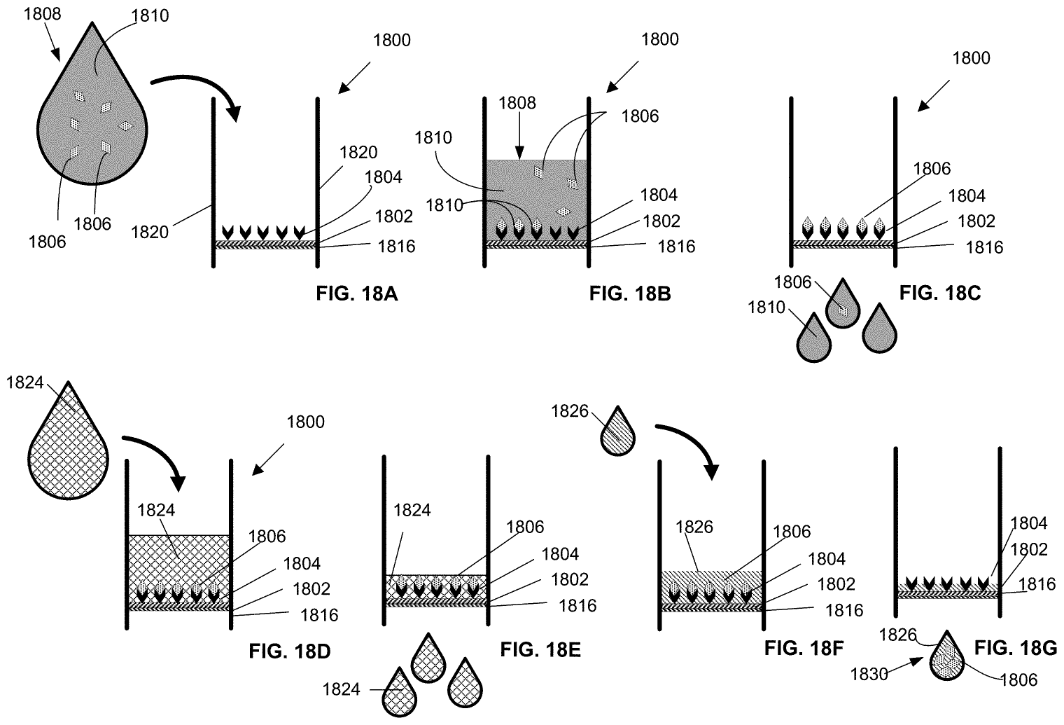

FIGS. 18A-18G depict use of a filtration device 1800 in which a capture ligand is immobilized on the 2-D surface of a stationary phase that includes a thin, substantially incompressible membrane. In the example of FIGS. 18A-18G, filtration device 1800 is a TB LAM filtration device in which a lectin is used as a capture ligand. It will be appreciated that such a device could be implemented with different capture ligands in order to capture other analytes. In an aspect, filtration device 1800 includes a stationary phase medium 1802 functionalized with at least one lectin 1804 adapted to bind a glycan of TB LAM to capture TB LAM 1806 from fluid sample 1808. Fluid sample 1808 includes TB LAM 1806 and a fluid component 1810. Filtration device 1800 includes a sieve element 1816 having openings small enough to block passage of the stationary phase medium 1802 but large enough to permit passage of unbound TB LAM 1806 and the fluid component 1810. Sieve element 1816 is formed of a mildly hydrophilic, chemically inert material having minimal nonspecific binding to components of the fluid sample.

In an aspect, TB LAM filtration device 1800 includes a housing 1820 configured to receive sieve element 1816 and stationary phase medium 1802. For example, housing 1820 may be a simple cup-like structure, as depicted in cross-section in FIGS. 18A-18G. Alternatively, housing 1820 may be similar in form to a syringe filter, e.g., similar to filtration-concentration device 430 depicted in FIG. 4D. In an aspect, filtration device 1800 includes a connector adapted to connect to the housing to a downstream vacuum source (not shown, but similar to fluid outlet port 450 in FIG. 4D, for example). In an aspect, TB LAM filtration device 1800 includes a connector adapted to connect to the housing to a upstream positive pressure source (not shown, but similar to inlet port 426 in FIG. 4D, for example). In an aspect, housing 1820 is configured for orienting sieve element 1816 and stationary phase medium 1802 such that gravity draws fluid through sieve element 1816 and away from stationary phase medium 1802. Housing 1820 may be so configured by including features that allow it to be placed on a support surface or attached to a supporting structure such as a receptacle for connecting waste fluid. Such features could include, for example, a level base region or several legs or tabs that support it with respect to the support surface, or a threaded region that permits it to be screwed onto corresponding threads on a jar or similar fluid receptacle.

In an aspect, stationary phase medium 1802 has a bed volume of between about 50 μl and about 400 μl. In an aspect, stationary phase medium 1802 has a bed volume less than about 200 μl. In an aspect, stationary phase medium 1802 has a bed volume less than about 300 μl.

In an aspect, stationary phase medium 1802 includes at least one of a resin, a gel, a hydrogel, a sponge, a fibrous material, a fiber mat, a cellulosic material, a cellulose pad, a polymer, a nanofiber, an electrospun polylactic acid, agarose, POROS® bioprocessing resin, Sepharose® gel filtration media, or Sephadex® gel filtration media. In an aspect, stationary phase medium 1802 includes a membrane, e.g. at least one of nitrocellulose, nylon, glass fiber, polytetrafluoroethylene, polyvinylidene difluoride, or Immunodyne® ABC membrane (available from PALL Corporation).

Sieve element 1816 can be constructed from a metal, a polymer, glass, fabric, a ceramic, a sintered material, or a felted material, for example. Sieve element 1816 should be chemically inert. Sieve element 1816 can take the form of a mesh, a plate having perforations therein, or a porous material.

In an aspect, lectin 1804 includes at least one lectin configured to bind TB LAM in a dose-dependent manner. In various aspects, lectin 1804 includes at least one lectin capable of binding specifically to mannose or at least one lectin capable of binding specifically to arabinose. For example, lectin 1804 may include Galanthus nivalis lectin, Hippeastrum hybrid lectin, or Lens culinaris agglutinin, or a combination thereof, e.g., a combination of Galanthus nivalis lectin and Hippeastrum hybrid lectin. In an aspect, lectin 1804 is configured (e.g., by appropriate choice of a lectin, such as the examples provided above) to release the analyte of interest under a mildly acidic condition (e.g., about pH 4). In an aspect, lectin 1804 is configured (e.g., by appropriate choice of a lectin, such as the examples provided above) to release the analyte of interest under a chaotropic condition. In an aspect, lectin 1804 is configured (e.g., by appropriate choice of a lectin, such as the examples provided above) to release the analyte of interest when exposed to an elution buffer compatible with downstream detection and quantification of the TB LAM with an α LAM-antibody.

Filtration device 1800 is used in a manner similar to that described and depicted in FIGS. 2A-2H. In FIG. 18A, fluid sample 1808 is added to filtration device 1800. In FIG. 18B, fluid sample 1808 is exposed to lectin 1804 in filtration device 1800 to allow TB LAM 1806 to bind to lectin 1804. Passing fluid sample 1808 through the stationary phase medium 1802 once may be sufficient to capture the TB LAM 1806. Alternatively, it may be preferable to pass fluid sample 1808 through stationary phase medium 1802 multiple times to provide sufficient opportunity for TB LAM 1806 to be captured.

In FIG. 18C, fluid component 1810 and unbound TB LAM 1806 pass through stationary phase medium 1802 and sieve element 1816, while TB LAM 1806 bound to lectin 1804 is retained on stationary phase medium 1802. In FIG. 18D, wash buffer 1824 is added to filtration device 1800. As shown in FIG. 18E, wash buffer 1824 passes through stationary phase medium 1802 and sieve element 1816, removing materials that contribute to background, while TB LAM 1806 bound to lectin 1804 remains on stationary phase medium 1802. As noted previously, wash buffer 1824 can be passed through stationary phase medium 1802 in multiple batches, allowing for the use of a large amount of wash buffer 1824 to accomplish thorough washing of TB LAM 1806. In FIGS. 18F and 18G, a small volume of elution buffer 1826 is added to filtration device 1800. TB LAM 1806 is eluted from lectin 1804 and passes through stationary phase medium 1802 and sieve element 1816, exiting filtration device 1800 as concentrated sample 1830, made up of TB LAM 1806 in elution buffer 1826. The final volume of concentrated sample 1830 is dependent upon the dead volume of the system, i.e., the amount of fluid that can be retained in stationary phase medium 1802.

Capture Concentration with Stationary Phase Functionalized Throughout its Bulk

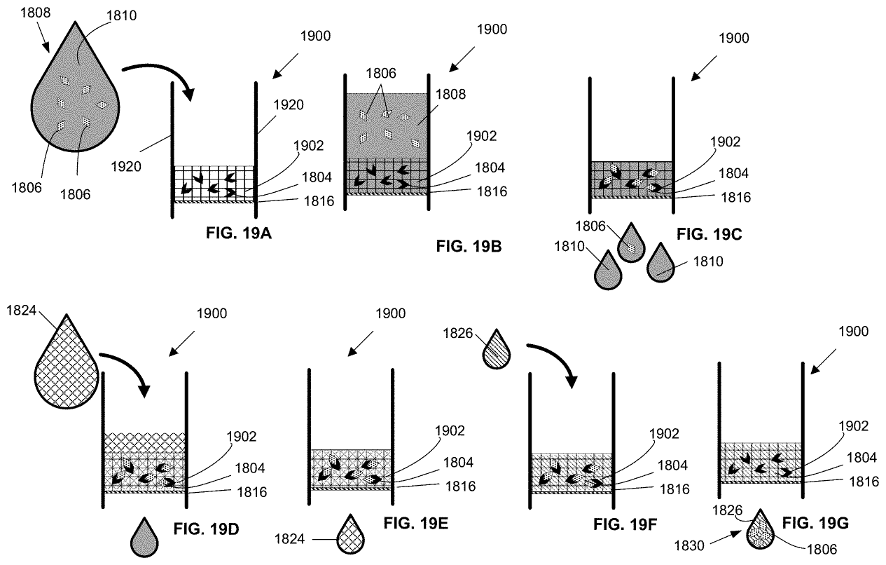

FIGS. 19A-19G, depict a filtration device 1900, similar to that depicted in FIGS. 18A-18G, however, in this case, the stationary phase medium 1902 includes an open pore capture bed, such as a sponge, with capture ligand immobilized in the 3-D bulk of the open pore capture bed. Again, the filtration device 1900 is illustrated as a TB LAM filtration device that uses a lectin 1804 (as described herein above) as a capture ligand, but the device could be implemented with different capture ligands in order to capture other analytes. In an aspect, filtration device 1900 includes a stationary phase medium 1902 functionalized with at least one lectin 1804 adapted to bind a glycan of TB LAM to capture TB LAM 1806 from fluid sample 1808 (which, as discussed above, includes TB LAM 1806 and a fluid component 1810). Filtration device 1900 includes a sieve element 1816 having openings small enough to block passage of the stationary phase medium 1902 but large enough to permit passage of unbound TB LAM 1806 and the fluid component 1810. Sieve element 1816 is formed of a mildly hydrophilic, chemically inert material having minimal nonspecific binding to components of the fluid sample. In an aspect, filtration device 1900 includes housing 1920, which may be similar to housing 1820 in FIG. 18A-18G, for example.

In an aspect, the stationary phase medium 1902 includes a resin such as agarose, POROS® bioprocessing resin (ThermoFisher Scientific), Sepharose® gel filtration Media (Millipor-Sigma), or Sephadex® gel filtration media (Millipor-Sigma), a gel, a hydrogel, a sponge, a fibrous material (e.g., a fiber mat), a cellulosic material (e.g., cellulose pad), a polymer, a nanofiber, electrospun polylactic acid, for example.

In an aspect, stationary phase medium 1902 includes a compressible structure, e.g. formed from, e.g., a porous material and/or a compressible material. For example, in an aspect a compressible structure can be formed from a porous material, a fibrous material, or an open cell foam, for example.

In FIG. 19A, fluid sample 1808 is added to filtration device 1900. In FIG. 19B, fluid sample 1808 is exposed to lectin 1804 in stationary phase medium 1902 to allow TB LAM 1806 to bind to lectin 1804. Passing fluid sample 1808 through the stationary phase medium 1902 once may be sufficient to capture the TB LAM 1806. Alternatively, it may be preferable to pass fluid sample 1808 through stationary phase medium 1902 multiple times to provide sufficient opportunity for TB LAM 1806 to be captured.

In FIG. 19C, fluid component 1810 and unbound TB LAM 1806 pass through stationary phase medium 1902 and sieve element 1816, while TB LAM 1806 bound to lectin 1804 is retained in stationary phase medium 1902. In FIG. 19D, wash buffer 1824 is added to filtration device 1900. In FIG. 19E, wash buffer 1824 passes through stationary phase medium 1902 and sieve element 1816, removing materials that contribute to background, while TB LAM 1806 bound to lectin 1804 remains in stationary phase medium 1902. As noted previously, wash buffer 1824 can be passed through stationary phase medium 1902 in multiple batches, allowing for the use of a large amount of wash buffer 1824 to accomplish thorough washing of TB LAM 1806. In FIGS. 19F and 19G, a small volume of elution buffer 1826 is added to filtration device 1900. TB LAM 1806 is eluted from lectin 1804 and passes through stationary phase medium 1902 and sieve element 1816, exiting filtration device 1900 as concentrated sample 1830, made up of TB LAM 1806 in elution buffer 1826. The final volume of concentrated sample 1830 is dependent upon the dead volume of the system, i.e., the amount of fluid that can be retained in stationary phase medium 1902.

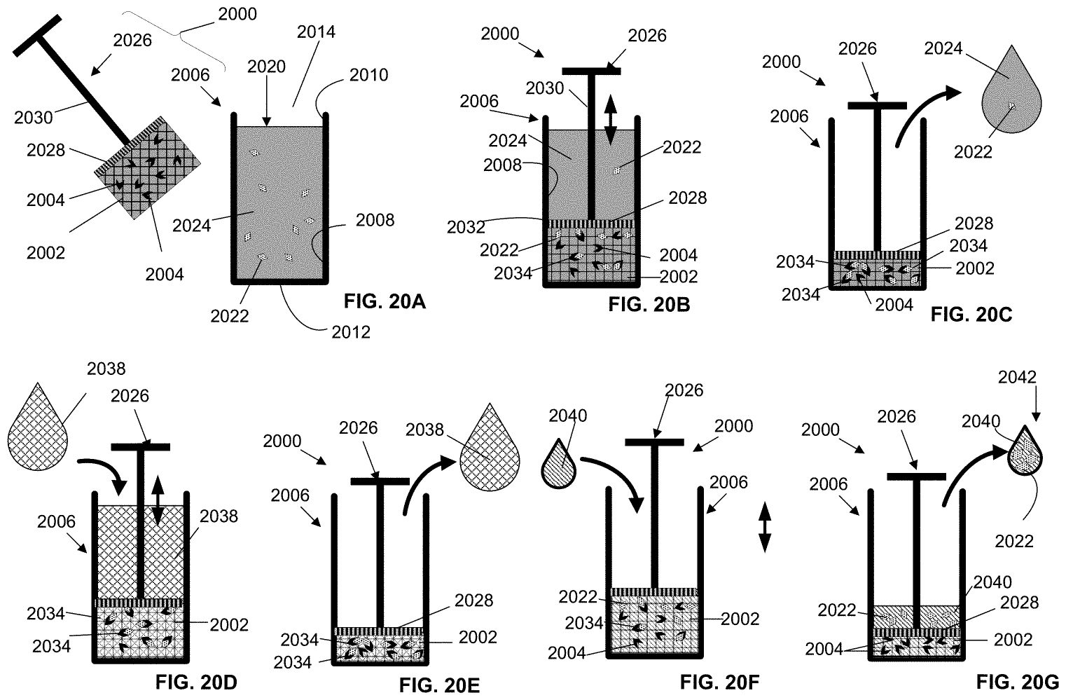

The dead volume of the system influences the final volume of the concentrated sample 1830. This approach is useful when the initial sample volume to be processed is much larger than that dead volume. If stationary phase medium 1902 is formed from a compressible material/structure, the dead volume can be reduced by compressing it, e.g. like squeezing out a sponge. Stationary phase medium 1902 can be kept in its uncompressed state while being contacted with fluid sample 1808, wash buffer 1824, or elution buffer 1826 (e.g. during steps depicted in FIGS. 19B, 19D, and 19F), but compressed during fluid removal steps (e.g., in FIGS. 19C, 19E, and 19G). FIGS. 20A-20G depict an example of a device that can be used in such an approach.

### Capture Concentration Device

FIGS. 20A-20G illustrates a capture concentration device 2000 which can be used in connection with stationary phase medium 2002 containing capture ligand 2004 immobilized within its 3-D bulk.

As shown in FIG. 20A, a capture concentration device 2000 includes a straight-walled container 2006, which has an interior surface 2008, a first end 2010, a second end 2012, and an opening 2014 at first end 2010. The straight-walled container 2006 is adapted to receive a fluid sample 2020 including an analyte of interest 2022 and a fluid component 2024. Capture concentration device 2000 also includes plunger 2026, which includes sieve element 2028 and shaft 2030. Sieve element 2028 is configured to support a stationary phase medium 2002, which is functionalized with at least one capture ligand 2004 adapted to bind an analyte of interest 2022 in the fluid sample 2020. Sieve element 2028 has openings small enough to block passage of stationary phase medium 2002 but large enough to permit passage of unbound analyte of interest 2022 and the fluid component 2024. As shown in FIG. 20B, sieve element 2028 is configured to slidably engage 2032 with the interior surface 2008 of straight-walled container 2006. Shaft 2030 is attached to sieve element 2028 and configured to transmit force to sieve element 2028 to drive sliding movement of sieve element 2028 within straight-walled container 2006. In an aspect, sieve element 2028 may be constructed from a mesh or screen-like material, a woven or felted material, or a rigid or semi-rigid plate or frame having apertures or openings formed therein. In an aspect, sieve element 2028 is configured to form a slidable seal with the interior surface 2008 of straight-walled container 2006, for example sieve element 2008 may include a gasket around its periphery, or its periphery may be sufficiently smooth to substantially seal with interior surface 2008. In an aspect, the slidable seal is a fluid-tight seal. In another aspect, the slidable seal blocks passage of stationary phase medium 2002 but may permit passage of fluid.

In an aspect, straight-walled container 2006 is substantially cylindrical. As depicted in FIGS. 20A-20G, straight-walled container 2006 is closed at second end 2012. In an alternative embodiment, opening 2014 at first end 2010 is a first opening, and the straight-walled container includes a second opening at the second end 2012 (not depicted in FIGS. 20A-20G), such that the straight walled container 2006 is similar in form to a syringe.

In an aspect, plunger 2026 is configured to support stationary phase medium 2002 on a side of sieve element 2028 facing toward second end 2012 of straight-walled container 2006 when plunger 2026 is positioned within straight-walled container 2006, as depicted in FIGS. 20A-20G. Thus, stationary phase medium 2002 is located between sieve element 2028 and second end 2012, such that, if stationary phase medium 2002 is compressible, it can be compressed between sieve element 2028 and second end 2012

Alternatively, in some embodiments, plunger 2026 is configured to support the stationary phase medium 2002 on a side of the sieve element 2028 facing toward the first end 2010 of the straight-walled container 2006 when plunger 2026 is positioned within straight-walled container 2006 (not illustrated).

In an aspect, sieve element 2028 includes a metal, polymer, ceramic, glass, or other material. In an aspect, one or both of sieve element 2028 and stationary phase medium 2002 are formed of a chemically inert material having minimal nonspecific binding to components of the fluid sample. In an aspect, the sieve element and the stationary phase medium are formed of mildly hydrophilic material. The stationary phase medium may take the form of, e.g., a disc of filter or sponge material that can be attached to the sieve element.

In an aspect, the stationary phase medium 2002 includes a compressible structure having the capture ligand 2004 immobilized within its bulk. In various aspects, the stationary phase medium includes a porous material and/or a compressible material. In an aspect, the stationary phase medium includes at least one of a resin, a gel, a hydrogel, a sponge, a fibrous material, a fiber mat, a cellulosic material, a cellulose pad), a polymer, a nanofiber, an electrospun polylactic acid, agarose, POROS® bioprocessing resin, Sepharose® gel filtration media, or Sephadex® gel filtration media.

In an aspect, the capture ligand 2004 includes at least one lectin, for example a lectin adapted to bind a glycan of TB LAM. For example, the lectin may be specific to mannos or arabinose. For example, the lectin may be Galanthus nivalis lectin, Hippeastrum hybrid lectin, or Lens culinaris agglutinin, for example as discussed herein above. In some aspects, the at least one lectin includes a combination of lectins, for example, a combination of Galanthus nivalis lectin and Hippeastrum hybrid lectin. The lectin may be configured to bind TB LAM in a dose-dependent manner.

As depicted in FIG. 20B, in use, plunger 2026 is inserted into container 2006 containing analyte 2022 in fluid component 2024. Exposure of capture ligand 2004 to analyte 2022 in fluid component 2024 is maximized by pushing the stationary phase medium 2002 back and forth through the sample, as indicated by the black arrow. After incubation with agitation, shown in FIG. 20B, plunger 2026 is depressed as shown in FIG. 20C, to compress stationary phase medium 2002. Fluid component 2024 and any unbound analyte 2022 is pressed out of stationary phase medium 2002, and passes through sieve element 2028 to the upper region of container 2006, where it can be poured off or pipetted out of container 2006. Analyte bound to capture ligand 2004, to form capture ligand-analyte complexes 2034, remains in stationary phase medium 2002. As shown in FIG. 20C, the volume of fluid component 2024 remaining in stationary phase medium 2002 is reduced due the compression of stationary phase medium 2002.

In FIG. 20D, wash buffer 2038 is added to container 2006 and plunger 2026 is moved up and down to move wash buffer 2038 through stationary phase medium 2002. In FIG. 20E, plunger 2026 is depressed to compress stationary phase medium 2002. Wash buffer 2038 and any interference-producing substances are pressed out of stationary phase medium 2002, and pass through sieve element 2028 to the upper region of container 2006, for removal. Capture ligand-analyte complexes 2034 remain in stationary phase medium 2002.

In FIG. 20F, elution buffer 2040 is added to container 2006 and plunger 2026 is moved up and down to move elution buffer 2040 through stationary phase medium 2002 to elute analyte 2022 from capture ligand 2004. In FIG. 20G, plunger 2026 is depressed to compress stationary phase medium 2002. Elution buffer 2040 and analyte 2022 are pressed out of stationary phase medium 2002, and pass through sieve element 2028 to the upper region of container 2006 for removal. In an aspect, the volume of elution buffer 2040 is smaller than the volume of fluid component 2024 in the original sample, such that cleaned analyte sample 2042 (analyte 2022 in elution buffer 2040) is concentrated relative to the original sample.

FIGS. 20A-20G depict a capture concentration device 2000 compresses stationary phase medium 2002 by direct manual application of pressure via plunger 2026. It will be appreciated that higher compression forces could be applied by modifying the device to include a simple mechanical advantage device if greater forces was needed.

FIGS. 20A-20G depicted a capture concentration device 2000 in which stationary phase medium 2002 is compressible. In related embodiments, a similar device can be constructed with a stationary phase medium that is a substantially incompressible membrane having the capture ligand immobilized on its surface. For example, incompressible membrane may be a nitrocellulose or polyvinylidene difluoride (PVDF) membrane.

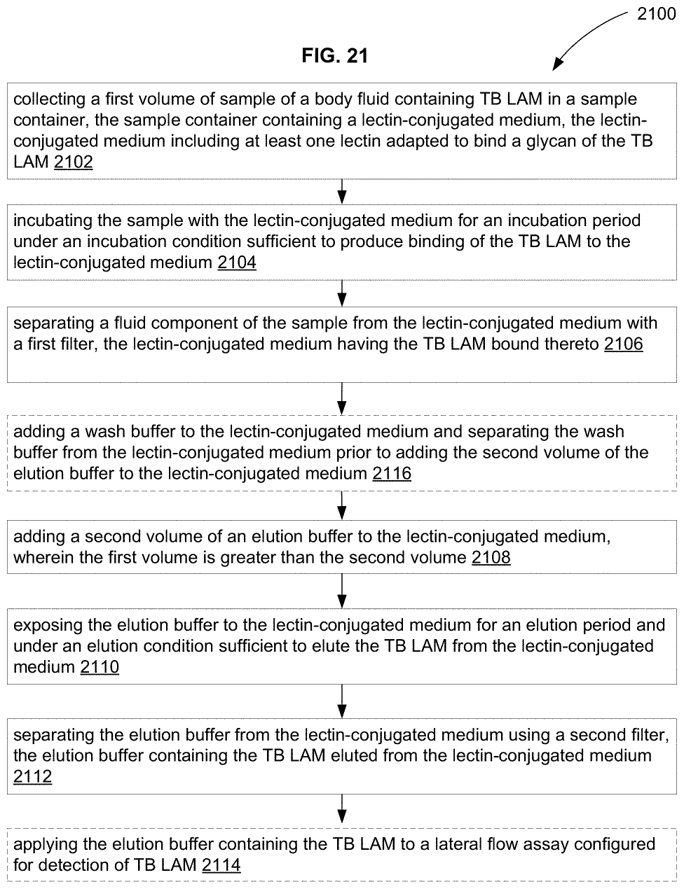

FIG. 21 depicts a generalized method of concentrating TB LAM. Method 2100 includes collecting a first volume of sample of a body fluid containing TB LAM in a sample container, the sample container containing a lectin-conjugated medium, the lectin-conjugated medium including at least one lectin adapted to bind a glycan of the TB LAM, at 2102; incubating the sample with the lectin-conjugated medium for an incubation period under an incubation condition sufficient to produce binding of the TB LAM to the lectin-conjugated medium, at 2104; separating a fluid component of the sample from the lectin-conjugated medium with a first filter, the lectin-conjugated medium having the TB LAM bound thereto, at 2106; adding a second volume of an elution buffer to the lectin-conjugated medium, wherein the first volume is greater than the second volume, at 2108; exposing the elution buffer to the lectin-conjugated medium for an elution period and under an elution condition sufficient to elute the TB LAM from the lectin-conjugated medium, at 2110; and separating the elution buffer from the lectin-conjugated medium using a second filter, the elution buffer containing the TB LAM eluted from the lectin-conjugated medium, at 2112. In an aspect, the incubation period is about 30 minutes, for example. It will be appreciated that in various aspects, lectin-containing medium can be added to the container before, after, or at the same time as the sample. The sample container may include a sample cup or a transfer container such as a jar, tube or vial, for example.

In some aspects, the lectin-conjugated medium includes at least one lectin configured to bind LAM in a dose-dependent manner. In various aspects, the lectin-conjugated medium includes, for example, at least one lectin capable of binding specifically to mannose (e.g., Galanthus nivalis lectin, Hippeastrum hybrid lectin, Lens culinaris agglutinin) or at least one lectin capable of binding specifically to arabinose. In an aspect, the lectin-conjugated medium includes a combination of Galanthus nivalis lectin and Hippeastrum hybrid lectin.

In an aspect, the method 2100 further includes applying the elution buffer containing the TB LAM to a lateral flow assay configured for detection of TB LAM, as indicated at 2114. For example, the lateral flow assay may be configured for antibody-based detection of TB LAM. In an aspect, the second volume (i.e., the volume of elution buffer) is between about 50 μl and about 400 μl. In some aspect, the second volume is less than about 200 μl, or less than about 300 μl.

In an aspect, method 2100 also includes a step of adding a wash buffer to the lectin-conjugated medium and separating the wash buffer from the lectin-conjugated medium prior to adding the second volume of the elution buffer to the lectin-conjugated medium, as indicated at 2116. This could be done between steps 2106 and 2108 in FIG. 21, for example.

In an aspect, the at least one lectin is configured to release the analyte of interest under a mildly acidic (e.g., about pH 4) condition. In an aspect, the at least one lectin is configured to release the analyte of interest under a chaotropic condition. In an aspect, the at least one lectin is configured to release the analyte of interest when exposed to an elution buffer compatible with downstream detection and quantification of the TB LAM with an α LAM-antibody. In an aspect, the elution condition includes a mildly acidic condition. In another aspect, the elution condition includes a chaotropic condition, which can be obtained by including at least one of urea, acetate, or MgCl₂ in the elution buffer. In various aspects, if elution buffer containing analyte is to be applied directly to a lateral flow assay for detection of TB LAM, the elution buffer is compatible with downstream detection and quantification of the TB LAM with an α LAM-antibody.

In various aspects, the lectin-conjugated medium includes a resin, a membrane, particles or beads. In some aspects, the lectin-conjugated medium includes a stationary phase medium functionalized with lectins.

In an aspect, separating the fluid component of the sample from the lectin-conjugated medium with the first filter, at 2106, includes applying the sample fluid with the lectin-conjugated medium to the first filter and causing the sample fluid to pass through the filter while the lectin-conjugated medium is retained on the first filter. Fluid can be caused to pass through the filter by applying vacuum to a downstream side of filter, applying pressure to an upstream side of filter, orienting the filter such that gravity draws fluid through filter, or drawing fluid through filter with capillary pressure, for example.

Similarly, in an aspect, separating the elution buffer from the lectin-conjugated medium with the second filter, at 2112, includes applying the elution buffer with the lectin-conjugated medium to the second filter and causing the elution buffer to pass through the second filter while the lectin-conjugated medium is retained on the second filter. Again, fluid can be caused to pass through the filter by applying vacuum to a downstream side of filter, applying pressure to an upstream side of filter, orienting the filter such that gravity draws fluid through filter, or drawing fluid through filter with capillary pressure, for example.

In various aspects, at least one of the first filter and the second filter includes an inline filter, a gravity filter, or a filter built into the sample container. In some aspects, the first filter is the same as the second filter. In other aspects, the first filter is distinct from the second filter.

### Prophetic Example Application 1: Lectin Beads in Urine Cup for Capture of TB-LAM

Devices and methods described herein can be used, for example, in the concentration and detection of TB-LAM. Mycoplasma-specific membrane glycolipid LAM is released into bodily fluids in small amounts in infected individuals with active TB infection. Isolation, detection and quantification of Mycoplasma LAM is important in diagnosing TB, for example using an immunoassay.

Concentration of LAM is necessary for its detection and quantification in high sensitivity assays. Mechanical concentration can be used but is labor- and equipment-intensive process that is not easy to integrate with lateral flow immunoassays. Therefore, a method can be formed that includes the following steps:
1. Collect urine sample having a volume of e.g. 10-50 ml in urine collection cup (see, e.g. FIG. 2A)
2. Allow the sample to incubate with GNL-conjugated beads for 30 minutes (see, e.g. FIGS. 2B-2C)
3. Remove the supernatant by running the sample through an inline filter to capture the beads (see, e.g. FIG. 2D)
4. Add small amount (e.g. 200 μL-300 μL) of elution and running buffer (see, e.g. FIG. 2G)
5. Transfer buffer containing eluted LAM (see, e.g. FIG. 2H) to lateral flow immunoassay for antibody-based detection (see FIG. 3A-3C)

Lectin from the snowdrop flower Galanthus nivalis Lectin (GNL) binds LAM in a dose-dependent manner. The kinetics of GNL-LAM interaction are comparable to activity of many antibodies, making the system feasible for use in assays. LAM can be removed (eluted) from GNL under mildly acidic (about pH 4) and chaotropic conditions (e.g. conditions that break hydrogen bonds, for example obtainable by using urea, acetate buffer, or MgCl₂ in the buffer). GNL binds mannose, and as an alternative, other mannose-specific lectins (such as Hippeastrum hybrid lectin (HHL)) can be used. For example, S. Mitra and H. R. Das described the use of a mannose specific lectin isolated from the fish Labeo rohita for detecting LAM in an ELISA format (Indian Journal of Clinical Biochemistry, 2001, 16(2), 181-184). Chinese Patent CN101974099A describes the use of flat lentil lectin to purify LAM to create a reagent suitable for detecting anti-LAM antibodies. Both references are incorporated herein by reference. In other aspects, arabinose-specific Lectins can be used to bind LAM. In some aspects, combinations of lectins can be used.

Isolation and purification of LAM using lectins according to methods described herein is cheap and compatible with the downstream immunodetection. Buffers used for both binding and elution of LAM are compatible with downstream detection and quantification of LAM using an α-LAM antibody, for example in a lateral flow assay.

### Prophetic Example Application 2

Devices and methods described herein can be used in various other applications involving washing and concentration of particular materials, as well. For example, the filtration-concentration device 430 depicted in FIGS. 4D-4M can be used to concentrate and wash parasite eggs in preparation for detection. For example, Schistosoma (blood flukes) are parasitic flatworms. Their eggs can be found in human feces. A stool sample could be diluted in a sample cup similar to container 406 in FIG. 4C, the diluted sample drawn up into e.g. syringe 420, and injected into filtration concentration device 430. Multiple syringe volumes can be injected into filtration concentration device for filtration, as needed. After initial filtration, wash solution can be injected into filtration concentration device 430, to wash the concentrated eggs. Following washing steps, concentrated eggs can be removed from upstream chamber 442 via retentate removal port 448, using a method similar to that depicted in FIGS. 4L and 4M.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

In a general sense, the various embodiments described herein can be implemented, individually and/or collectively, by various types of mechanical and/or electro-mechanical systems having a wide range of electrical components such as hardware, software, firmware, or virtually any combination thereof, and a wide range of components that can impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electro-mechanical systems include but are not limited to a variety of consumer electrical systems, as well as other systems such as motorized transport systems, factory automation systems, security systems, and communication/computing systems. Those skilled in the art will recognize that electro-mechanical as used herein is not necessarily limited to a system that has both electrical and mechanical actuation except as context can dictate otherwise.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, manually, or under control of a wide range of hardware, software, firmware, or virtually any combination thereof. In an embodiment, several portions of the subject matter described herein can be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the reader will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

The reader will recognize that the state of the art has progressed to the point where there is little distinction left between hardware and software implementations of—aspects of systems; the use of hardware or software is generally (but not always, in that in certain contexts the choice between hardware and software can become significant) a design choice representing cost vs. efficiency tradeoffs. The reader will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer can opt for a mainly hardware and/or firmware vehicle; alternatively, if flexibility is paramount, the implementer can opt for a mainly software implementation; or, yet again alternatively, the implementer can opt for some combination of hardware, software, and/or firmware. Hence, there are several possible vehicles by which the processes and/or devices and/or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which can vary. The reader will recognize that optical aspects of implementations will typically employ optically-oriented hardware, software, and or firmware.

In a general sense, the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). The subject matter described herein can be implemented in an analog or digital fashion or some combination thereof.

This disclosure has been made with reference to various example embodiments. However, those skilled in the art will recognize that changes and modifications can be made to the embodiments without departing from the scope of the present disclosure. For example, various operational steps, as well as components for carrying out operational steps, can be implemented in alternate ways depending upon the particular application or in consideration of any number of cost functions associated with the operation of the system; e.g., one or more of the steps can be deleted, modified, or combined with other steps.

Additionally, as will be appreciated by one of ordinary skill in the art, principles of the present disclosure, including components, can be reflected in a computer program product on a computer-readable storage medium having computer-readable program code means embodied in the storage medium. Any tangible, non-transitory computer-readable storage medium can be utilized, including magnetic storage devices (hard disks, floppy disks, and the like), optical storage devices (CD-ROMs, DVDs, Blu-ray discs, and the like), flash memory, and/or the like. These computer program instructions can be loaded onto a general purpose computer, special purpose computer, or other programmable data processing apparatus to produce a machine, such that the instructions that execute on the computer or other programmable data processing apparatus create a means for implementing the functions specified. These computer program instructions can also be stored in a computer-readable memory that can direct a computer or other programmable data processing apparatus to function in a particular manner, such that the instructions stored in the computer-readable memory produce an article of manufacture, including implementing means that implement the function specified. The computer program instructions can also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

The herein described components (e.g., steps), devices, and objects and the discussion accompanying them are used as examples for the sake of conceptual clarity. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural and/or singular terms herein, the reader can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

In some instances, one or more components can be referred to herein as “configured to.” The reader will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications can be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense the convention (e.g., “a system having at least one of A, B, or C” would include but not be limited to systems that have A alone, B alone, C alone, A and B together, A and C together, B and C together, and/or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” With respect to the appended claims, the recited operations therein can generally be performed in any order. Examples of such alternate orderings can include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

Clause 1. A method of concentrating TB LAM, comprising:

collecting a first volume of sample of a body fluid containing TB LAM in a sample container, the sample container containing a lectin-conjugated medium, the lectin-conjugated medium including at least one lectin adapted to bind a glycan of the TB LAM;

incubating the sample with the lectin-conjugated medium for an incubation period under an incubation condition sufficient to produce binding of the TB LAM to the lectin-conjugated medium;

separating a fluid component of the sample from the lectin-conjugated medium with a first filter, the lectin-conjugated medium having the TB LAM bound thereto; adding a second volume of an elution buffer to the lectin-conjugated medium, wherein the first volume is greater than the second volume;

exposing the elution buffer to the lectin-conjugated medium for an elution period and under an elution condition sufficient to elute the TB LAM from the lectin-conjugated medium;

separating the elution buffer from the lectin-conjugated medium using a second filter, the elution buffer containing the TB LAM eluted from the lectin-conjugated medium.

Clause 2. The method of clause 1, further comprising applying the elution buffer containing the TB LAM to a lateral flow assay configured for detection of TB LAM.

Clause 3. The method of clause 2, wherein the lateral flow assay is configured for antibody-based detection of TB LAM.

Clause 4. The method of clause 1, further comprising adding a wash buffer to the lectin-conjugated medium and separating the wash buffer from the lectin-conjugated medium prior to adding the second volume of the elution buffer to the lectin-conjugated medium.

Clause 5. The method of clause 1, wherein the lectin-conjugated medium includes at least one lectin configured to bind LAM in a dose-dependent manner.

Clause 6. The method of clause 1, wherein the lectin-conjugated medium includes at least one lectin capable of binding specifically to mannose.

Clause 7. The method of clause 1, wherein the lectin-conjugated medium includes at least one lectin capable of binding specifically to arabinose.

Clause 8. The method of clause 1, wherein the lectin-conjugated medium includes Galanthus nivalis lectin.

Clause 9. The method of clause 1, wherein the lectin-conjugated medium includes Hippeastrum hybrid lectin.

Clause 10. The method of clause 1, wherein the lectin-conjugated medium includes Lens culinaris agglutinin.

Clause 11. The method of clause 1, wherein the lectin-conjugated medium includes a combination of Galanthus nivalis lectin and Hippeastrum hybrid lectin.

Clause 12. The method of clause 1, wherein the lectin-conjugated medium includes at least one of a resin, a gel, a hydrogel, a sponge, a fibrous material, a fiber mat, a cellulosic material, a cellulose pad, a polymer, a nanofiber, an electrospun polylactic acid, agarose, POROS® bioprocessing resin, Sepharose® gel filtration media, or Sephadex® gel filtration media.

Clause 13. The method of clause 1, wherein the lectin-conjugated medium includes a membrane.

Clause 14. The method of clause 1, wherein the lectin-conjugated medium includes particles or beads.

Clause 15. The method of clause 14, wherein separating the fluid component of the sample from the lectin-conjugated medium the first filter includes applying the sample fluid with the lectin-conjugated medium to the first filter and causing the sample fluid to pass through the filter while the lectin-conjugated medium is retained on the first filter.

Clause 16. The method of clause 14, wherein separating the elution buffer from the lectin-conjugated medium with the second filter includes applying the elution buffer with the lectin-conjugated medium to the second filter and causing the elution buffer to pass through the second filter while the lectin-conjugated medium is retained on the second filter.

Clause 17. The method of clause 1, wherein the lectin-conjugated medium includes a stationary phase medium functionalized with lectins.

Clause 18. The method of clause 1, wherein the incubation period is about 30 minutes.

Clause 19. The method of clause 1, wherein the elution condition includes a mildly acidic condition.

Clause 20. The method of clause 1, wherein the elution condition includes a chaotropic condition.

Clause 21. The method of clause 20, wherein chaotropic condition is obtained by including at least one of urea, acetate or MgCl₂ in the elution buffer.

Clause 22. The method of clause 1, wherein the elution buffer is compatible with downstream detection and quantification of the LAM with an α LAM-antibody.

Clause 23. The method of clause 1, wherein the sample container includes a sample cup.

Clause 24. The method of clause 1, wherein the sample container includes a transfer container.

Clause 25. The method of clause 1, wherein separating the fluid component of the sample from the lectin-conjugated medium with the first filter includes applying the sample fluid with the lectin-conjugated medium to the first filter and causing the sample fluid to pass through the first filter while the lectin-conjugated medium is retained on the first filter.

Clause 26. The method of clause 1, wherein separating the elution buffer from the lectin-conjugated medium with the second filter, includes applying the elution buffer with the lectin-conjugated medium to the second filter and causing the elution buffer to pass through the second filter while the lectin-conjugated medium is retained on the second filter.

Clause 27. The method of clause 1, wherein at least one of the first filter and the second filter includes an inline filter.

Clause 28. The method of clause 1, wherein at least one of the first filter and the second filter includes a gravity filter.

Clause 29. The method of clause 1, wherein at least one of the first filter and the second filter includes a filter built into the sample container.

Clause 30. The method of clause 1, wherein the first filter is the same as the second filter.

Clause 31. The method of clause 1, wherein the first filter is distinct from the second filter.

Clause 32. The method of clause 1, wherein the second volume is between about 50 μl and about 400 μl.

Clause 33. The method of clause 1, wherein the second volume is less than about 200 μl.

Clause 34. The method of clause 1, wherein the second volume is less than about 300 μl.

Clause 35. A sample filtration container, comprising:

a base defining a bottom of the sample filtration container;

at least one side wall contiguous with the base, the at least one side wall enclosing an interior of the sample filtration container;

an opening at a top of the sample filtration container, the opening adapted to receive a sample including a fluid component and a particulate material carried in the fluid component;

a divider located within the interior of the sample filtration container and dividing the interior of the sample filtration container into an upper portion and a lower portion, the divider including a size exclusion filter, wherein the size exclusion filter has a first side communicating with the upper portion of the sample filtration container and a second side communicating with the lower portion of the sample filtration container, wherein the size exclusion filter has a pore size adapted to allow passage of the fluid component of the sample while blocking passage of the particulate material; and

a capillary medium within the lower portion of the sample filtration container, the capillary medium adapted to draw the fluid component of the sample through the size exclusion filter from the upper portion to the lower portion of the sample filtration container.

Clause 36. The sample filtration container of clause 35, further comprising a sample collection region located at a bottom of the upper portion of the sample container, the sample collection region having a reduced cross-sectional area relative to the upper portion as a whole.

Clause 37. The sample filtration container of clause 36, wherein the divider is substantially planar and oriented at an oblique angle relative to the at least one side wall, and wherein the sample collection region is bounded by the at least one side wall and the divider at a location at which the divider forms an acute angle with the at least one side wall.

Clause 38. The sample filtration container of clause 36, wherein the divider includes a shaped depression, and wherein the sample collection region is located at a bottom of the shaped depression in the divider.

Clause 39. The sample filtration container of clause 35, wherein the capillary medium is adapted to retain the fluid component within the lower portion of the sample container.

Clause 40. The sample filtration container of clause 35, wherein the capillary medium is adapted to retain a fluid volume of between about 10 ml and about 50 ml.

Clause 41. The sample filtration container of clause 35, wherein the capillary medium includes a cellulosic material, a fiber-based material, a glass fiber, a sponge, a resin, a superabsorbent polymer, or a hydrogel.

Clause 42. The sample filtration container of clause 35, wherein the size exclusion filter has a pore size of between about 0.1 μm and about 0.4 μm.

Clause 43. The sample filtration container of clause 35, wherein the size exclusion filter includes at least one of organic membranes made up of one or more polymers, as cellulose, nitrocellulose, cellulose acetate, polysulfone, polyvinylidene fluoride, polyethersulfone, polyethylene sulfone, polyamide, polyethylene teraphthalate, or polytetrafluoroethylene, or glass fiber.

Clause 44. A method of filtering a sample, comprising:

adding a sample including a fluid component and a particulate material into a sample filtration container, the sample filtration container including

a base defining a bottom of the sample filtration container;

at least one side wall contiguous with the base, the at least one side wall enclosing an interior of the sample container;

a divider located within the interior of the sample container and dividing the interior of the sample container into an upper portion and a lower portion, the divider including a size exclusion filter, wherein the size exclusion filter has a first side communicating with the upper portion of the sample container and a second side communicating with the lower portion of the sample container, wherein the size exclusion filter has a pore size adapted to allow passage of the fluid component of the sample while blocking passage of the particulate material;

a sample collection region located at a bottom of the upper portion of the sample container; and

a capillary medium within the lower portion of the sample container, the capillary medium adapted to draw the fluid component of the sample through the size exclusion filter from the upper portion to the lower portion of the sample container;

allowing the fluid component of the sample to be drawn through the size exclusion filter and into the capillary medium; and

removing filtrate including the particulate material from the sample collection region located at a bottom of the upper portion of the sample container.

Clause 45. A lateral flow assay device, comprising:

a loading region adapted to receive a fluid containing a functionalized nanoparticle-captured analyte complex including one or more functionalized nanoparticle and an analyte of interest in a carrier fluid, the loading region including

a sample pad; and

a filter element overlying the sample pad, wherein the filter element includes pores small enough to block passage of the functionalized nanoparticle through the filter element but large enough to permit passage of the carrier fluid and unbound analyte of interest through the filter element to the sample pad; and

a lateral flow membrane downstream of the sample pad and including one or more capture components adapted to capture the analyte of interest.

Clause 46. The lateral flow assay device of clause 45, wherein the one or more capture components include one or more capture component adapted to capture TB LAM.

Clause 47. The lateral flow assay device of clause 45, wherein the one or more capture components include one or more antibody adapted to capture TB LAM.

Clause 48. The lateral flow assay device of clause 45, wherein lateral flow membrane includes a test line including the one or more capture components.

Clause 49. The lateral flow assay device of clause 45, wherein the filter element has a pore size of between about 0.1 μm and about 0.4 μm.

Clause 50. The lateral flow assay device of clause 45, wherein the filter element is formed of a chemically inert material having minimal nonspecific binding to components of the fluid.

Clause 51. The lateral flow assay device of clause 45, wherein the filter element is formed of a mildly hydrophilic material.

Clause 52. A method of detecting a biomarker of interest from fluid sample, comprising:

collecting a fluid sample including a fluid component containing a biomarker;

incubating the fluid sample with a plurality of functionalized nanoparticles for an incubation period sufficient to produce binding of at least a portion of the biomarker with the functionalized nanoparticles to form functionalized nanoparticle-captured biomarker complexes, the functionalized nanoparticles including nanoparticles functionalized with one or more ligands having an affinity to the biomarker;

filtering the fluid sample and functionalized nanoparticle-captured biomarker complexes with a size-exclusion filter to separate the functionalized nanoparticle-captured biomarker complexes from the fluid component;

transferring the functionalized nanoparticle-captured biomarker complexes to a lateral flow assay device; and

detecting the biomarker at a test line of the lateral flow assay device, the test line including antibodies specific to the biomarker and adapted to bind the biomarker at the test line.

Clause 53. The method of clause 52 including washing the functionalized nanoparticle-captured biomarker complexes on the size-exclusion filter with a wash buffer prior to transferring the functionalized nanoparticle-captured biomarker complexes to the lateral flow assay device.

Clause 54. The method of clause 52, wherein the lateral flow assay device is a half-strip lateral flow assay device, and wherein detecting the biomarker at the test line includes detecting the functionalized nanoparticle-biomarker complex at the test line, wherein the functionalized nanoparticle includes a detectable component.

Clause 55. The method of clause 52, including eluting the biomarker from the functionalized nanoparticle on the lateral flow assay device.

Clause 56. The method of clause 55, wherein the lateral flow assay device includes a size exclusion filter overlying a sample pad, the exclusion filter having pore size sufficient to permit passage of the biomarker but not the functionalized nanoparticle, and wherein transferring the functionalized nanoparticle-captured biomarker complexes to the lateral flow assay device includes applying the functionalized nanoparticle-captured biomarker complexes to the size exclusion filter.

Clause 57. The method of clause 55, including exposing the eluted biomarker to an antibody specific to the biomarker on the lateral flow assay device, wherein the antibody specific to the biomarker is conjugated to a detectable component, and wherein detecting the biomarker at a test line includes detecting the detectable component.

Clause 58. The method of clause 55, wherein the lateral flow assay device includes a filter element overlying an absorbent pad, the filter element supported by a movable framework and having pore size sufficient to permit passage of fluid and unbound analyte but not the functionalized nanoparticle, and wherein transferring the functionalized nanoparticle-captured biomarker complexes to the lateral flow assay device includes applying the functionalized nanoparticle-captured biomarker complexes to the filter element; and wherein the method further includes

allowing excess fluid associated with the functionalized nanoparticle-captured biomarker complexes to pass through first filter element and be absorbed by the absorbent pad; and

transferring the filter element on the movable framework to a sample pad of the lateral flow assay device;

wherein eluting the biomarker from the functionalized nanoparticle includes eluting the biomarker into the sample pad through the filter element.

Clause 59. The method of clause 55, wherein the lateral flow assay device includes a first filter element overlying an absorbent pad, the first filter element supported by a movable framework attached to a hinge element, the first filter element and having pore size sufficient to permit passage of fluid but not the functionalized nanoparticle, and wherein transferring the functionalized nanoparticle-captured biomarker complexes to the lateral flow assay device includes applying the functionalized nanoparticle-captured biomarker complexes to the first filter element; and wherein the method further includes

allowing excess fluid associated with the functionalized nanoparticle-captured biomarker complexes to pass through the first filter element and be absorbed by the absorbent pad; and

moving the first filter element from a position over the absorbent pad to a position over a second filter element overlying a sample pad of the lateral flow assay device by rotating the movable framework around an axis of the hinge element to flip the first filter element over;

wherein eluting the biomarker from the functionalized nanoparticle includes applying an elution buffer to the first filter element and allowing the elution buffer to pass through the first filter element, eluting the biomarker into the sample pad through the second filter element.

Clause 60. A lateral flow assay device, comprising:

a support layer;

an absorbent pad disposed on the support layer;

a movable framework configured to fit closely and removably over the absorbent pad;

a first filter element supported by the movable framework, the first filter element configured for fluid communication with the absorbent pad through one or more apertures in the movable framework, wherein the first filter element includes pores small enough to block passage of a functionalized nanoparticle-captured analyte complex through the first filter element but large enough to permit passage of a carrier fluid through the first filter element to the absorbent pad;

a sample pad supported by the support layer, wherein the sample pad is configured so that the movable framework can be fit closely over the sample pad; and

a lateral flow membrane downstream of the sample pad and including one or more capture components specific to the analyte of interest.

Clause 61. The lateral flow assay device of clause 60, wherein the first filter element includes at least one of organic membranes made up of one or more polymers, as cellulose, nitrocellulose, cellulose acetate, polysulfone, polyvinylidene fluoride, polyethersulfone, polyethylene sulfone, polyamide, polyethylene teraphthalate, or polytetrafluoroethylene, or glass fiber.

Clause 62. The lateral flow assay device of clause 60, wherein the first filter element includes pores large enough to permit passage of unbound analyte through the first filter element to the absorbent pad.

Clause 63. The lateral flow assay device of clause 60, wherein the first filter element has a pore size of between about 0.1 μm and about 0.4 μm.

Clause 64. The lateral flow assay device of clause 60, wherein the first filter element is formed of a chemically inert material having minimal nonspecific binding to components of the fluid sample.

Clause 65. The lateral flow assay device of clause 60, wherein the first filter element is formed of a mildly hydrophilic material.

Clause 66. The lateral flow assay device of clause 60, wherein the one or more capture components include one or more capture component adapted to capture TB LAM.

Clause 67. The lateral flow assay device of clause 60, wherein the one or more capture components include one or more antibody adapted to capture TB LAM.

Clause 68. The lateral flow assay device of clause 60, wherein lateral flow membrane includes a test line including the one or more capture components.

Clause 69. The lateral flow assay device of clause 60, wherein the movable framework is configured to fit over the sample pad with the first filter element in fluid communication with the sample pad through one or more apertures in the movable framework.

Clause 70. The lateral flow assay device of clause 77, wherein the movable framework is configured to slide from the absorbent pad to the sample pad.

Clause 71. The lateral flow assay device of clause 60, further comprising

a second filter element located over the sample pad, wherein the second filter element includes pores small enough to block passage of the functionalized nanoparticle-captured analyte complex and functionalized nanoparticle through the second filter element but large enough to permit passage of a second carrier fluid and unbound analyte through the second filter element to the sample pad; and

a hinge element disposed on the support layer between the absorbent pad and the sample pad and attached to the moveable framework such that the moveable framework can be rotated around an axis of the hinge element to flip the first filter element over to move the first filter element from a position over the absorbent pad to a position over the second filter element, so that the first filter element is in fluid communication with the sample pad through the second filter element.

Clause 72. The lateral flow assay device of clause 71, wherein at least one of the first filter element and the second filter element includes at least one of organic membranes made up of one or more polymers, as cellulose, nitrocellulose, cellulose acetate, polysulfone, polyvinylidene fluoride, polyethersulfone, polyethylene sulfone, polyamide, polyethylene teraphthalate, or polytetrafluoroethylene, or glass fiber.

Clause 73. The lateral flow assay device of clause 71, wherein the second filter element has a pore size of between about 0.1 μm and about 0.4 μm.

Clause 74. The lateral flow assay device of clause 71, wherein the second filter element is formed of a chemically inert material having minimal nonspecific binding to components of the fluid sample.

Clause 75. The lateral flow assay device of clause 71, wherein the second filter element is formed of a mildly hydrophilic material.

Clause 76. A filtration-concentration device, comprising:
a filter membrane having a first side and a second side, the filter membrane having pores small enough to block passage of a functionalized nanoparticle-captured analyte complex from the first side to the second side but large enough to permit passage of fluid or unbound analyte from the first side to the second side;
a housing configured to contain the filter membrane, the housing having an upstream chamber in fluid communication with the first side of the filter membrane and downstream chamber in fluid communication with the second side of the filter membrane;
an inlet port in fluid communication with the upstream chamber, the inlet port adapted to receive a fluid sample containing a functionalized nanoparticle-captured analyte complex in a first volume of the fluid;
a fluid outlet port in fluid communication with the downstream chamber, the fluid outlet port configured to permit fluid including a portion of the first volume of fluid to exit the filtration concentration device; and
a retentate removal port in communication with the upstream chamber, the retentate removal port configured to allow removal of a retentate from the upstream chamber;
wherein the filter membrane is chemically inert with respect to the functionalized nanoparticle-captured analyte complex and the fluid and exhibits little or no non-specific binding to materials in the fluid;

wherein the upstream chamber has a volume sufficient to contain a second volume of fluid, wherein the second volume is less than the first volume.

Clause 77. The filtration-concentration device of clause 84, wherein the filter membrane includes at least one of an organic membrane, a polymer, cellulose, nitrocellulose, cellulose acetate, polysulfone, polyvinylidene fluoride, polyethersulfone, polyethylene sulfone, polyamide, polyethylene teraphthalate, or polytetrafluoroethylene, or glass fiber.

Clause 78. The filtration-concentration device of clause 84, wherein the fluid outlet port is adapted for connection to a vacuum source.

Clause 79. The filtration-concentration device of clause 84, further comprising a vacuum source connected downstream of the fluid outlet port.

Clause 80. The filtration-concentration device of clause 84, wherein the inlet port is adapted for connection to a positive pressure source.

Clause 81. The filtration-concentration device of clause 84, further comprising a positive pressure source connected upstream of the inlet port.

Clause 82. A capture concentration device, comprising:
a straight-walled container having an interior surface, a first end, a second end, and an opening at the first end, the straight-walled container adapted to receive a fluid sample including an analyte of interest and a fluid component; and
a plunger including
   a sieve element configured to slidably engage with the interior surface of the straight-walled container and to support a stationary phase medium functionalized with at least one capture ligand adapted to bind an analyte of interest in the fluid sample, the sieve element having openings small enough to block passage of the stationary phase medium but large enough to permit passage of unbound analyte of interest and the fluid component; and

a shaft attached to the sieve element and configured to transmit force to the sieve element to drive sliding movement of the sieve element within the straight-walled container.

Clause 83. The capture concentration device of clause 82, wherein the straight-walled container is substantially cylindrical.

Clause 84. The capture concentration device of clause 82, wherein the straight-walled container is closed at the second end.

Clause 85. The capture concentration device of clause 82, wherein the opening at the first end is a first opening, and wherein the straight-walled container includes a second opening at the second end.

Clause 86. The capture concentration device of clause 82, wherein the plunger is configured to support the stationary phase medium on a side of the sieve element facing toward the first end of the straight-walled container when the plunger is positioned within the straight-walled container.

Clause 87. The capture concentration device of clause 82, wherein the plunger is configured to support the stationary phase medium on a side of the sieve element facing toward the second end of the straight-walled container when the plunger is positioned within the straight-walled container

Clause 88. The capture concentration device of clause 82, wherein the sieve element includes a metal, a polymer, glass, fabric, a ceramic, a sintered material, or a felted material.

Clause 89. The capture concentration device of clause 82, wherein the sieve element and the stationary phase medium are formed of a chemically inert material having minimal nonspecific binding to components of the fluid sample.

Clause 90. The capture concentration device of clause 82, wherein the sieve element and the stationary phase medium are formed of mildly hydrophilic material.

Clause 91. The capture concentration device of clause 82, further comprising the stationary phase medium.

Clause 92. The capture concentration device of clause 82, wherein the stationary phase medium is formed of a chemically inert material having minimal nonspecific binding to components of the fluid sample.

Clause 93. The capture concentration device of clause 82, wherein the stationary phase medium includes a substantially incompressible membrane having the capture ligand immobilized on its surface.

Clause 94. The capture concentration device of clause 93, wherein the stationary phase medium includes at least one of nitrocellulose, nylon, glass fiber, polytetrafluoroethylene, polyvinylidene difluoride, or Immunodyne® ABC membrane.

Clause 95. The capture concentration device of clause 82, wherein the stationary phase medium includes a compressible structure having the capture ligand immobilized within its bulk.

Clause 96. The capture concentration device of clause 82, wherein the second volume is between about 50 μl and about 400 μl.

Clause 97. The capture concentration device of clause 82, wherein the second volume is less than about 200 μl.

Clause 98. The capture concentration device of clause 82, wherein the second volume is less than about 300 μl.

Clause 99. The capture concentration device of clause 82, wherein the stationary phase medium includes a porous material.

Clause 100. The capture concentration device of clause 82, wherein the stationary phase medium includes a compressible material.

Clause 101. The capture concentration device of clause 82, wherein the capture ligand includes at least one lectin.

Clause 102. The capture concentration device of clause 101, wherein the at least one lectin is adapted to bind a glycan of TB LAM.

Clause 103. The capture concentration device of clause 101, wherein the at least one lectin includes Galanthus nivalis lectin.

Clause 104. The capture concentration device of clause 101, wherein the at least one lectin includes Hippeastrum hybrid lectin.

Clause 105. The capture concentration device of clause 101, wherein the at least one lectin includes Lens culinaris agglutinin.

Clause 106. The capture concentration device of clause 101, wherein the at least one lectin includes a combination of Galanthus nivalis lectin and Hippeastrum hybrid lectin.

Clause 107. The capture concentration device of c clause 101, wherein the at least one lectin includes at least one lectin configured to bind LAM in a dose-dependent manner.

Clause 108. The capture concentration device of clause 101, wherein the at least one lectin includes at least one lectin capable of binding specifically to mannose.

Clause 109. The capture concentration device of clause 101, wherein the at least one lectin includes at least one lectin capable of binding specifically to arabinose.

Clause 110. The capture concentration device of clause 101, wherein the at least one lectin is configured to release the analyte of interest under a mildly acidic condition.

Clause 111. The capture concentration device of clause 101, wherein the at least one lectin is configured to release the analyte of interest under a chaotropic condition.

Clause 112. The capture concentration device of clause 101, wherein the at least one lectin is configured to release the analyte of interest when exposed to an elution buffer compatible with downstream detection and quantification of the LAM with an α LAM-antibody.

Clause 113. A TB LAM filtration device, comprising:

a stationary phase medium functionalized with at least one lectin adapted to bind a glycan of TB LAM to capture TB LAM from a fluid sample, the fluid sample including the TB LAM and a fluid component; and

a sieve element having openings small enough to block passage of the stationary phase medium but large enough to permit passage of unbound TB LAM and the fluid component, wherein the sieve element is formed of a mildly hydrophilic, chemically inert material having minimal nonspecific binding to components of the fluid sample.

Clause 114. The TB LAM filtration device of clause 113, wherein the stationary phase medium includes a compressible structure having the at least one lectin immobilized within its bulk.

Clause 115. The TB LAM filtration device of clause 114, wherein the compressible structure includes a porous material.

Clause 116. The TB LAM filtration device of clause 114, wherein the compressible structure includes a compressible material.

Clause 117. The TB LAM filtration device of clause 113, wherein the stationary phase medium includes a substantially incompressible membrane having the at least one lectin immobilized on its surface.

Clause 118. The TB LAM filtration device of clause 117, wherein the substantially incompressible membrane includes at least one of nitrocellulose, nylon, glass fiber, polytetrafluoroethylene, polyvinylidene difluoride, or Immunodyne® ABC membrane.

Clause 119. The TB LAM filtration device of clause 113, wherein the stationary phase medium includes at least one of a resin, a gel, a hydrogel, a sponge, a fibrous material, a fiber mat, a cellulosic material, a cellulose pad, a polymer, a nanofiber, an electrospun polylactic acid, agarose, POROS® bioprocessing resin, Sepharose® gel filtration media, or Sephadex® gel filtration media.

Clause 120. The TB LAM filtration device of clause 113, wherein the sieve element includes a metal, a polymer, glass, fabric, a ceramic, a sintered material, or a felted material.

Clause 121. The TB LAM filtration device of clause 113, wherein the at least one lectin includes at least one lectin configured to bind LAM in a dose-dependent manner.

Clause 122. The TB LAM filtration device of clause 113, wherein the at least one lectin includes at least one lectin capable of binding specifically to mannose.

Clause 123. The TB LAM filtration device of clause 113, wherein the at least one lectin includes at least one lectin capable of binding specifically to arabinose.

Clause 124. The TB LAM filtration device of clause 113, wherein the at least one lectin includes Galanthus nivalis lectin.

Clause 125. The TB LAM filtration device of clause 113, wherein the at least one lectin includes Hippeastrum hybrid lectin.

Clause 126. The TB LAM filtration device of clause 113, wherein the at least one lectin includes Lens culinaris agglutinin.

Clause 127. The TB LAM filtration device of clause 113, wherein the at least one lectin includes a combination of Galanthus nivalis lectin and Hippeastrum hybrid lectin.

Clause 128. The TB LAM filtration device of clause 113, wherein the at least one lectin is configured to release the analyte of interest under a mildly acidic condition.

Clause 129. The TB LAM filtration device of clause 113, wherein the at least one lectin is configured to release the analyte of interest under a chaotropic condition.

Clause 130. The TB LAM filtration device of clause 113, wherein the at least one lectin is configured to release the analyte of interest when exposed to an elution buffer compatible with downstream detection and quantification of the LAM with an α LAM-antibody.

Clause 131. The TB LAM filtration device of clause 113, further comprising a housing configured to receive the sieve element and the stationary phase medium.

Clause 132. The TB LAM filtration device of clause 131, including a connector adapted to connect to the housing to a downstream vacuum source.

Clause 133. The TB LAM filtration device of clause 131, including a connector adapted to connect to the housing to a upstream positive pressure source.

Clause 134. The TB LAM filtration device of clause 131, wherein the housing is configured to for orienting the sieve element and stationary phase medium such that gravity draws fluid through the sieve element and away from the stationary phase medium.

1. A sample filtration container, comprising:
a base defining a bottom of the sample filtration container;
at least one side wall contiguous with the base, the at least one side wall enclosing an interior of the sample filtration container;
an opening at a top of the sample filtration container, the opening adapted to receive a sample including a fluid component and a particulate material carried in the fluid component;
a divider located within the interior of the sample filtration container and dividing the interior of the sample filtration container into an upper portion and a lower portion, the divider including a size exclusion filter, wherein the size exclusion filter has a first side communicating with the upper portion of the sample filtration container and a second side communicating with the lower portion of the sample filtration container, wherein the size exclusion filter has a pore size adapted to allow passage of the fluid component of the sample while blocking passage of the particulate material; and
a capillary medium within the lower portion of the sample filtration container, the capillary medium adapted to draw the fluid component of the sample through the size exclusion filter from the upper portion to the lower portion of the sample filtration container.

2.-11. (canceled)

12. A lateral flow assay device, comprising:
a loading region adapted to receive a fluid containing a functionalized nanoparticle-captured analyte complex including one or more functionalized nanoparticle and an analyte of interest in a carrier fluid, the loading region including
a sample pad; and
a filter element overlying the sample pad, wherein the filter element includes pores small enough to block passage of the functionalized nanoparticle through the filter element but large enough to permit passage of the carrier fluid and unbound analyte of interest through the filter element to the sample pad; and
a lateral flow membrane downstream of the sample pad and including one or more capture components adapted to capture the analyte of interest.

13. The lateral flow assay device of claim 12, wherein the one or more capture components include one or more capture component adapted to capture TB LAM.

14. The lateral flow assay device of claim 12, wherein the one or more capture components include one or more antibody adapted to capture TB LAM.

15. The lateral flow assay device of claim 12, wherein lateral flow membrane includes a test line including the one or more capture components.

16. The lateral flow assay device of claim 12, wherein the sample pad includes at least one of cellulose, glass fiber, cotton, rayon, a woven mesh, and a synthetic non-woven material.

17. The lateral flow assay device of claim 12, wherein the filter element has a pore size of between about 0.1 μm and about 0.4 μm.

18. The lateral flow assay device of claim 12, wherein the filter element is formed of a chemically inert material having minimal nonspecific binding to components of the fluid.

19. The lateral flow assay device of claim 12, wherein the filter element is formed of a mildly hydrophilic material.

20. A lateral flow assay device, comprising:
a support layer;
an absorbent pad disposed on the support layer;
a movable framework configured to fit closely and removably over the absorbent pad;
a first filter element supported by the movable framework, the first filter element configured for fluid communication with the absorbent pad through one or more apertures in the movable framework, wherein the first filter element includes pores small enough to block passage of a functionalized nanoparticle-captured analyte complex through the first filter element but large enough to permit passage of a carrier fluid through the first filter element to the absorbent pad;
a sample pad supported by the support layer, wherein the sample pad is configured so that the movable framework can be fit closely over the sample pad; and
a lateral flow membrane downstream of the sample pad and including one or more capture components specific to the analyte of interest.

21.-36. (canceled)

37. A filtration-concentration device, comprising:
a filter membrane having a first side and a second side, the filter membrane having pores small enough to block passage of a functionalized nanoparticle-captured analyte complex from the first side to the second side but large enough to permit passage of fluid or unbound analyte from the first side to the second side;
a housing configured to contain the filter membrane, the housing having an upstream chamber in fluid communication with the first side of the filter membrane and downstream chamber in fluid communication with the second side of the filter membrane;
an inlet port in fluid communication with the upstream chamber, the inlet port adapted to receive a fluid sample containing a functionalized nanoparticle-captured analyte complex in a first volume of the fluid;
a fluid outlet port in fluid communication with the downstream chamber, the fluid outlet port configured to permit fluid including a portion of the first volume of fluid to exit the filtration concentration device; and
a retentate removal port in communication with the upstream chamber, the retentate removal port configured to allow removal of a retentate from the upstream chamber;
wherein the filter membrane is chemically inert with respect to the functionalized nanoparticle-captured analyte complex and the fluid and exhibits little or no non-specific binding to materials in the fluid;
wherein the upstream chamber has a volume sufficient to contain a second volume of fluid, wherein the second volume is less than the first volume.

38.-42. (canceled)

43. A capture concentration device, comprising:
a straight-walled container having an interior surface, a first end, a second end, and an opening at the first end, the straight-walled container adapted to receive a fluid sample including an analyte of interest and a fluid component; and
a plunger including
a sieve element configured to slidably engage with the interior surface of the straight-walled container and to support a stationary phase medium functionalized with at least one capture ligand adapted to bind an analyte of interest in the fluid sample, the sieve element having openings small enough to block passage of the stationary phase medium but large enough to permit passage of unbound analyte of interest and the fluid component; and
a shaft attached to the sieve element and configured to transmit force to the sieve element to drive sliding movement of the sieve element within the straight-walled container.

44. The capture concentration device of claim 43, wherein the straight-walled container is substantially cylindrical.

45. The capture concentration device of claim 43, wherein the sieve element is configured to form a slidable seal with the interior surface of the straight-walled container.

46. The capture concentration device of claim 43, wherein the straight-walled container is closed at the second end.

47. The capture concentration device of claim 43, wherein the opening at the first end is a first opening, and wherein the straight-walled container includes a second opening at the second end.

48. The capture concentration device of claim 43, wherein the plunger is configured to support the stationary phase medium on a side of the sieve element facing toward the first end of the straight-walled container when the plunger is positioned within the straight-walled container.

49. The capture concentration device of claim 43, wherein the plunger is configured to support the stationary phase medium on a side of the sieve element facing toward the second end of the straight-walled container when the plunger is positioned within the straight-walled container

50. The capture concentration device of claim 43, wherein the sieve element includes a metal, a polymer, glass, fabric, a ceramic, a sintered material, or a felted material.

51.-52. (canceled)

53. The capture concentration device of claim 43, further comprising the stationary phase medium.

54.-61. (canceled)

62. The capture concentration device of claim 53, wherein the stationary phase medium includes a porous material having the capture ligand immobilized within its bulk.

63. The capture concentration device of claim 53, wherein the stationary phase medium includes a compressible material having the capture ligand immobilized within its bulk.

64. The capture concentration device of claim 53, wherein the capture ligand includes at least one lectin.

65.-75. (canceled)

76. A TB LAM filtration device, comprising:
a stationary phase medium functionalized with at least one lectin adapted to bind a glycan of TB LAM to capture TB LAM from a fluid sample, the fluid sample including the TB LAM and a fluid component; and
a sieve element having openings small enough to block passage of the stationary phase medium but large enough to permit passage of unbound TB LAM and the fluid component, wherein the sieve element is formed of a mildly hydrophilic, chemically inert material having minimal nonspecific binding to components of the fluid sample.

77.-97. (canceled)

98. The capture concentration device of claim 43, wherein the sieve element and the stationary phase medium are formed of at least one of a chemically inert material having minimal nonspecific binding to components of the fluid sample, or mildly hydrophilic material.

99. The capture concentration device of claim 53, wherein the stationary phase medium is formed of a chemically inert material having minimal nonspecific binding to components of the fluid sample; wherein the stationary phase medium includes a substantially incompressible membrane having the capture ligand immobilized on its surface; wherein the stationary phase medium includes at least one of nitrocellulose, nylon, glass fiber, polytetrafluoroethylene, polyvinylidene difluoride, or Immunodyne® ABC membrane; wherein the stationary phase medium includes a compressible structure having the capture ligand immobilized within its bulk; or wherein the stationary phase medium includes at least one of a resin, a gel, a hydrogel, a sponge, a fibrous material, a fiber mat, a cellulosic material, a cellulose pad, a polymer, a nanofiber, an electrospun polylactic acid, agarose, POROS® bioprocessing resin, Sepharose® gel filtration media, or Sephadex® gel filtration media.

100. The capture concentration device of claim 43, wherein the second volume is less than about 200 μl, less than about 300 μl, or between about 50 μl and about 400 μl.

101. The capture concentration device of claim 64, wherein the at least one lectin includes at least one of a lectin adapted to bind a glycan of TB LAM, Galanthus nivalis lectin, Hippeastrum hybrid lectin, Lens culinaris agglutinin, a combination of Galanthus nivalis lectin and Hippeastrum hybrid lectin, at least one lectin configured to bind LAM in a dose-dependent manner, at least one lectin capable of binding specifically to mannose, or at least one lectin capable of binding specifically to arabinose.

102. The capture concentration device of claim 64, wherein the at least one lectin is configured to release the analyte of interest under a mildly acidic condition, under a chaotropic condition, or when exposed to an elution buffer compatible with downstream detection and quantification of the LAM with an α LAM-antibody.
